SEQUENCE LISTING (8 sequences)

SEQ ID NO: 1 - protein, 5 aa, Murine
DYAMH

SEQ ID NO: 2 - protein, 17 aa, Murine
LISIYSDHTNYNQKFQG

SEQ ID NO: 3 - protein, 12 aa, Murine
NNYGNYGWYFDV

SEQ ID NO: 4 - protein, 10 aa, Murine
SASSSVSYMH

SEQ ID NO: 5 - protein, 7 aa, Murine
DTSKLAS

SEQ ID NO: 6 - protein, 9 aa, Murine
FQGSGYPYT

SEQ ID NO: 7 - protein, 121 aa, Artificial Sequence
  Artificially Synthesized Sequence
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 8 - protein, 106 aa, Artificial Sequence
  Artificially Synthesized Sequence
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

(19) United States
(12) Patent Application Publication
(10) Pub. No.: US 2021/0155903 A1
(43) Pub. Date: May 27, 2021

(54) Combination Therapy

(71) Applicant: GlaxoSmithKline Intellectual Property Development Limited, Brentford Middlesex (GB)

(72) Inventors: Meixia BI, Collegeville, PA (US); Christopher B. HOPSON, Collegeville, PA (US)

(73) Assignee: GLAXOSMITHKLINE INTELLECTUAL PROPERTY DEVELOPMENT LIMITED

(21) Appl. No.: 17/168,551

(22) Filed: Feb. 5, 2021

Related U.S. Application Data
(62) Division of application No. 15/749,984, filed on Feb. 2, 2018.
(60) Provisional application No. 62/202,217, filed on Aug. 7, 2015.
(60) Provisional application No. 62/218,030, filed on Sep. 14, 2015.

(51) Int. Cl. C12N 5/0783; C07K 16/28; A61P 35/00; A61K 38/17; A61K 45/06

(52) U.S. Cl. CPC C12N 5/0646; C07K 16/2818; A61P 35/00; A61K 2039/507; A61K 45/06; C12N 5/0638; A61K 38/1774

(57) ABSTRACT

Methods for increasing expression of at least one co-stimulatory and/or co-inhibitory receptor on a T cell comprising contacting said T cell with an anti-CTLA4 antibody are provided. In one aspect the co-stimulatory and/or co-inhibitory receptor is selected from the group of: PD-1, OX40, ICOS, CD137, TIM3, and LAG3. The present invention also provides methods of treating cancer in a human in need thereof comprising administering an anti-CTLA antibody and at least one additional agent directed to at least one co-stimulatory and/or co-inhibitory receptor to said human. In one aspect, the agent is directed to at least one co-stimulatory and/or co-inhibitory receptor selected from the group of: PD-1, OX40, ICOS, CD137 (4-1BB), TIM3, and LAG3.

### CROSS REFERENCE TO RELATED APPLICATIONS

This application is a divisional of U.S. application Ser. No. 15/749,984, filed on Feb. 2, 2018, which is a section 371 of International Application No. PCT/IB2016/054725, filed on Aug. 4, 2016, which claims the benefit of U.S. Provisional 62/202,217 filed on Aug. 7, 2015 and U.S. Provisional 62/218,030 filed on Sep. 14, 2015, all of which are incorporated herein in their entireties.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Jan. 29, 2021, is named PU65948D1_Sequence_Listing and is 4 KB in size.

### FIELD OF THE INVENTION

The present invention relates generally to immunotherapy in the treatment of human disease. More specifically, the present invention relates to the use of immunomodulators in the treatment of cancer.

### BACKGROUND OF THE INVENTION

Enhancing anti-tumor T cell function and inducing T cell proliferation is a powerful and new approach for cancer treatment. Three immune-oncology antibodies (e.g., immuno-modulators) are presently marketed. Anti-CTLA-4 (YERVOY/ipilimumab) is thought to augment immune responses at the point of T cell priming and anti-PD-1 antibodies (OPDIVO/nivolumab and KEYTRUDA/pembrolizumab) are thought to act in the local tumor microenvironment, by relieving an inhibitory checkpoint in tumor specific T cells that have already been primed and activated.

ICOS is a co-stimulatory T cell receptor with structural and functional relation to the CD28/CTLA-4-Ig superfamily (Hutloff, et al., “ICOS is an inducible T-cell co-stimulator structurally and functionally related to CD28”, Nature, 397: 263-266 (1999)). Emerging data from patients treated with anti-CTLA4 antibodies also point to the positive role of ICOS+ effector T cells in mediating an anti-tumor immune response. Patients with metastatic melanoma (Giacomo A M D et al., “Long-term survival and immunological parameters in metastatic melanoma patients who respond to ipilimumab 10 mg/kg within an expanded access program”, Cancer Immunol Immunother., 62(6); 1021-8 (2013)); urothelial (Carthon B C et al., “Preoperative CTLA-4 blockade: Tolerability and immune monitoring in the setting of a presurgical clinical trial” Clin Cancer Res., 16(10); 2861-71 (2010)); breast (Vonderheide R H et al., “Tremelimumab in combination with exemestane in patients with advanced breast cancer and treatment-associated modulation of inducible costimulator expression on human T cells”, Clin Cancer Res., 16(13); 3485-94 (2010)); and prostate cancer which have increased absolute counts of circulating and tumor infiltrating CD4⁺ICOS⁺ and CD8⁺ICOS⁺ T cells after ipilimumab treatment have significantly better treatment related outcomes than patients where little or no increases are observed. Importantly, it was shown that ipilimumab changes the COS⁺ T effector:T_{reg} ratio, reversing an abundance of T_{regs} pre-treatment to a significant abundance of T effectors vs. T_{regs} following treatment (Liakou C I et al., “CTLA-4 blockade increases IFN-gamma producing CD4+ICOShi cells to shift the ratio of effector to regulatory T cells in cancer patients”, Proc Natl Acad Sci USA. 105(39); 14987-92 (2008)) and (Vonderheide R H et al., “Tremelimumab in combination with exemestane in patients with advanced breast cancer and treatment-associated modulation of inducible costimulator expression on patient T cells”, Clin Cancer Res., 16(13); 3485-94 (2010)). Therefore, ICOS positive T effector cells are a positive predictive biomarker of ipilimumab response which points to the potential advantage of activating this population of cells with an agonist ICOS antibody.

Thus, there is a need for combination treatments of immunomodulators for the treatment of disease, in particular cancer.

### BRIEF DESCRIPTION OF THE DRAWINGS

FIG. 1: Phenotype of A2058 tumour infiltrating lymphocytes in donor 6943. Single cell suspensions were prepared from tumors recovered post Ipilimumab treatment. Phenotype of tumor infiltrating lymphocytes (TILs) was analyzed by flow cytometry. Higher frequency of total human CD45, CD3 T lymphocytes in both CD4/CD8+ population was observed in TIL samples from Ipilimumab treated tumors. Data demonstrated Ipilimumab treatment increase the lymphocytes infiltration to tumor.

FIG. 2: Phenotype of A2058 tumor infiltrating lymphocytes in donor 7814. Single cell suspensions were prepared from tumors recovered post Ipilimumab treatment. Phenotype of tumor infiltrating lymphocytes (TILs) was analyzed by flow cytometry. Higher frequency of total human CD45, CD3 T lymphocytes in both CD4/CD8+ population was observed in TIL samples from 2 out of 3 Ipilimumab treated tumors. Data demonstrated Ipilimumab treatment increase the lymphocytes infiltration to tumor.

FIG. 3: Ipilimumab treatment augments T-cell activation. Mouse blood samples were collected weekly. T cell activation of human PBMC was analyzed by flow cytometry. Expanded human PBMC were mainly CD3⁺ T cells, and Ipilimumab treatment increased the frequency of both CD4⁺ and CD8⁺ T cells. Ipilimumab upregulates expression of PD-1, OX40, ICOS, CD137,TIM3 and LAG3 on CD4+ or CD8+ or both T cell populations.

FIG. 4: T cell activation assessment of A2058 tumor infiltrating lymphocytes in donor 7814. Single cell suspensions were prepared from tumors recovered post Ipilimumab treatment. T cell activation of tumor infiltrating lymphocytes (TILs) was analyzed by flow cytometry. Similar to corresponding peripheral blood data, Tumor Infiltrating Lymphocytes (TILs) in A2058 tumors were mainly CD3⁺ T cells, and Ipilimumab treatment increased the frequency of both CD4⁺ and CD8⁺ T cells. Ipilimumab upregulate expression of PD-1, OX40, ICOS, CD137, TIM3 and LAG3 on CD4+ or CD8+ or both T cell populations.

FIG. 5: H2L5 hIgG4PE in combination with ipilimumab results increased proinflammatory cytokine production as compared to single antibody treatment in PBMC pre-stimulation assay.

FIG. 6: H2L5 hIgG4PE in combination with pembrolizumab results increased proinflammatory cytokine production as compared to single antibody treatment in PBMC pre-stimulation assay.

FIG. 7: H2L5 hIgG4PE plus ipilimumab combination induces increased proinflamatory cytokine production in a modified MLR assay with CEFT peptide and pre-incubation.

FIG. 8: H2L5 hIgG4PE plus pembrolizumab combination induces increased proinflamatory cytokine production in a modified MLR assay with CEFT peptide and pre-incubation.

FIG. 9: H2L5 hIgG4PE anti-ICOS agonist mAb alone and in combination with pembrolizumab results in tumor growth inhibition in a human PBMC A2058 Melanoma mouse tumor model.

FIG. 10: anti-ICOS murine surrogate mAb results in significant tumor growth inhibition and increased survival in combination with an anti-PD1 murine surrogate mAb in the CT26 mouse tumor model.

FIG. 11: anti-ICOS murine surrogate mAb results in significant tumor growth inhibition and increased survival in combination with an anti-PD1 murine surrogate mAb in the EMT6 mouse tumor model.

### SUMMARY OF THE INVENTION

In one embodiment the present invention provides methods for increasing expression of at least one co-stimulatory and/or co-inhibitory receptor on a circulating T cell comprising contacting said T cell with an anti-CTLA4 antibody. In one aspect the co-stimulatory and/or co-inhibitory receptor is selected from the group of: PD-1, OX40, ICOS, CD137, TIM3, and LAG3.

In one embodiment, methods are provided for treating cancer in a human in need thereof comprising administering an anti-CTLA antibody and at least one additional agent directed to at least one co-stimulatory and/or co-inhibitory receptor to said human. In one aspect, the agent is directed to at least one co-stimulatory and/or co-inhibitory receptor selected from the group of: PD-1, OX40, ICOS, CD137 (4-1BB), TIM3, and LAG3.

In one embodiment, an anti-CTLA4 antibody for use in treating cancer in a human in need thereof is provided, wherein the CTLA4 antibody is administered with at least one additional agent directed to at least one co-stimulatory and/or co-inhibitory receptor in said human, wherein administration of the CTLA4 antibody increases expression of said at least one co-stimulatory and/or co-inhibitory receptor in said human. In one aspect, the agent is directed to at least one co-stimulatory and/or co-inhibitory receptor selected from the group of: PD-1, OX40, ICOS, CD137, TIM3, and LAG3.

In one embodiment, an anti-CTLA4 antibody and at least one additional agent directed to at least one co-stimulatory and/or co-inhibitory receptor in a human for simultaneous or sequential use in treating cancer in a human in need thereof are provided, wherein administration of the CTLA4 antibody increases expression of said at least one co-stimulatory and/or co-inhibitory receptor in said human. In one aspect, the agent is directed to at least one co-stimulatory and/or co-inhibitory receptor selected from the group of: PD-1, OX40, ICOS, CD137, TIM3, and LAG3.

### DETAILED DESCRIPTION OF THE INVENTION

### Definitions

As used herein “ICOS” means any Inducible T-cell costimulator protein. Pseudonyms for ICOS (Inducible T-cell COStimulator) include AILIM; CD278; CVID1, JTT-1 or JTT-2, MGC39850, or 8F4. ICOS is a CD28-superfamily costimulatory molecule that is expressed on activated T cells. The protein encoded by this gene belongs to the CD28 and CTLA-4 cell-surface receptor family. It forms homodimers and plays an important role in cell-cell signaling, immune responses, and regulation of cell proliferation. Human ICOS is a 199 amino acid protein (Accession No.: UniProtKB-Q9Y6W8 (ICOS_HUMAN).

Activation of ICOS occurs through binding by ICOS-L (B7RP-1/B7-H2). Neither B7-1 nor B7-2 (ligands for CD28 and CTLA4) bind or activate ICOS. However, ICOS-L has been shown to bind weakly to both CD28 and CTLA-4 (Yao S et al., “B7-H2 is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naïve T_{H}1 and T_{H}2 effector T cell populations (Paulos C M et al., “The inducible costimulator (ICOS) is critical for the development of human Th17 cells”, Sci Transl Med, 2(55); 55ra78 (2010)). ICOS expression is highly induced on CD4⁺ and CD8+ effector T cells following activation through TCR engagement (Wakamatsu E, et al., “Convergent and divergent effects of costimulatory molecules in conventional and regulatory CD4⁺ T cells”, Proc Natal Acad Sci USA, 110(3); 1023-8 (2013)). Co-stimulatory signalling through ICOS receptor only occurs in T cells receiving a concurrent TCR activation signal (Sharpe A H and Freeman G J. “The B7-CD28 Superfamily”, Nat. Rev Immunol, 2(2); 116-26 (2002)). In activated antigen specific T cells, ICOS regulates the production of both T_{H}1 and T_{H}2 cytokines including IFN-γ, TNF-α, IL-10, IL-4, IL-13 and others. ICOS also stimulates effector T cell proliferation, albeit to a lesser extent than CD28 (Sharpe A H and Freeman G J. “The B7-CD28 Superfamily”, Nat. Rev Immunol, 2(2); 116-26 (2002)). Antibodies to ICOS and methods of using in the treatment of disease are described, for instance, in WO 2012/131004, US20110243929, and US20160215059. US20160215059 is incorporated by reference herein. Combination treatment of anti-CTLA4 antibodies and ICOS-ligand and anti-ICOS antibodies are described in US 2012251556.

In one embodiment, the ICOS antibodies of the present invention comprise any one or a combination of the following CDRs:

CDRH1:
(SEQ ID NO: 1)
DYAMH
CDRH2:
(SEQ ID NO: 2)
LISIYSDHTNYNQKFQG
CDRH3:
(SEQ ID NO: 3)
NNYGNYGWYFDV
CDRL1:
(SEQ ID NO: 4)
SASSSVSYMH
CDRL2:
(SEQ ID NO: 5)
DTSKLAS
CDRL3:
(SEQ ID NO: 6)
FQGSGYPYT

In some embodiments, the anti-ICOS antibodies of the present invention comprise a heavy chain variable region having at least 90% sequence identity to SEQ ID NO:7. Suitably, the ICOS binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:7.

### Humanized Heavy Chain (V_{H}) Variable Region (H2):

(SEQ ID NO: 7)
QVQLVQSGAE VKKPGSSVKV SCKASGYTFT DYAMHWVRQA
PGQGLEWMGL ISIYSDHTNY NQKFQGRVTI TADKSTSTAY
MELSSLRSED TAVYYCGRNN YGNYGWYFDV WGQGTTVTVS
S

In one embodiment of the present invention the ICOS antibody comprises CDRL1 (SEQ ID NO:4), CDRL2 (SEQ ID NO:5), and CDRL3 (SEQ ID NO:6) in the light chain variable region having the amino acid sequence set forth in SEQ ID NO:8. ICOS binding proteins of the present invention comprising the humanized light chain variable region set forth in SEQ ID NO:8 are designated as “L5.” Thus, an ICOS binding protein of the present invention comprising the heavy chain variable region of SEQ ID NO:7 and the light chain variable region of SEQ ID NO:8 can be designated as H2L5 herein.

In some embodiments, the ICOS binding proteins of the present invention comprise a light chain variable region having at least 90% sequence identity to the amino acid sequence set forth in SEQ ID NO:8. Suitably, the ICOS binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:8.

### Humanized Light Chain (V_{L}) Variable Region (L5)

(SEQ ID NO: 8)
EIVLTQSPAT LSLSPGERAT LSCSASSSVS YMHWYQQKPG
QAPRLLIYDT SKLASGIPAR FSGSGSGTDY TLTISSLEPE
DFAVYYCFQG SGYPYTFGQG TKLEIK

CDRs or minimum binding units may be modified by at least one amino acid substitution, deletion or addition, wherein the variant antigen binding protein substantially retains the biological characteristics of the unmodified protein, such as an antibody comprising SEQ ID NO:7 and SEQ ID NO:8.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acID Typically, the modification is a substitution, particularly a conservative substitution, for example as shown in Table 1 below.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence | Gly, Pro |
| chain orientation | |
| Aromatic | Trp, Tyr, Phe |

The subclass of an antibody in part determines secondary effector functions, such as complement activation or Fc receptor (FcR) binding and antibody dependent cell cytotoxicity (ADCC) (Huber, et al., Nature 229(5284): 419-20 (1971); Brunhouse, et al., Mol Immunol 16(11): 907-17 (1979)). In identifying the optimal type of antibody for a particular application, the effector functions of the antibodies can be taken into account. For example, hIgG1 antibodies have a relatively long half life, are very effective at fixing complement, and they bind to both FcγRI and FcγRII. In contrast, human IgG4 antibodies have a shorter half life, do not fix complement and have a lower affinity for the FcRs. Replacement of serine 228 with a proline (S228P) in the Fc region of IgG4 reduces heterogeneity observed with hIgG4 and extends the serum half life (Kabat, et al., “Sequences of proteins of immunological interest” 5.sup.th Edition (1991); Angal, et al., Mol Immunol 30(1): 105-8 (1993)). A second mutation that replaces leucine 235 with a glutamic acid (L235E) eliminates the residual FcR binding and complement binding activities (Alegre, et al., J Immunol 148(11): 3461-8 (1992)). The resulting antibody with both mutations is referred to as IgG4PE. The numbering of the hIgG4 amino acids was derived from EU numbering reference: Edelman, G. M. et al., Proc. Natl. Acad. USA, 63, 78-85 (1969). PMID: 5257969. In one embodiment of the present invention the ICOS antibody is an IgG4 isotype. In one embodiment, the ICOS antibody comprises an IgG4 Fc region comprising the replacement S228P and L235E may have the designation IgG4PE.

As used herein “ICOS-L” and “ICOS Ligand” are used interchangeably and refer to the membrane bound natural ligand of human ICOS. ICOS ligand is a protein that in humans is encoded by the ICOSLG gene. ICOSLG has also been designated as CD275 (cluster of differentiation 275). Pseudonyms for ICOS-L include B7RP-1 and B7-H2.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al. (2002) Curr. Opin. Immunol 14:391779-82; Bennett et al. (2003) J Immunol 170:711-8) The initial members of the family, CD28 and ICOS, were discovered by functional effects on augmenting T cell proliferation following the addition of monoclonal antibodies (Hutloff et al. (1999) Nature 397:263-266; Hansen et al. (1980) Immunogenics 10:247-260). PD-1 was discovered through screening for differential expression in apototic cells (Ishida et al. (1992) EMBO J 11:3887-95) The other members of the family, CTLA-4, and BTLA were discovered through screening for differential expression in cytotoxic T lymphocytes and TH1 cells, respectively. CD28, ICOS and CTLA-4 all have an unpaired cysteine residue allowing for homodimerization. In contrast, PD-1 is suggested to exist as a monomer, lacking the unpaired cysteine residue characteristic in other CD28 family members. PD-1 antibodies and methods of using in treatment of disease are described in U.S. Pat. Nos. 7,595,048; 8,168,179; 8,728,474; 7,722,868; 8,008,449; 7,488,802; 7,521,051; 8,088,905; 8,168,757; 8,354,509; and US Publication Nos. US20110171220; US20110171215; and US20110271358. Combinations of CTLA-4 and PD-1 antibodies are described in U.S. Pat. No. 9,084,776.

Opdivo/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

KEYTRUDA/pembrolizumab is an anti-PD-1 antibodies marketed for the treatment of lung cancer by Merck. The amino acid sequence of pembrolizumab and methods of using are disclosed in U.S. Pat. No. 8,168,757.

CD134, also known as OX40, is a member of the TNFR-superfamily of receptors which is not constitutively expressed on resting naïve T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. OX40/OX40-ligand (OX40 Receptor)/(OX40L) are a pair of costimulatory molecules critical for T cell proliferation, survival, cytokine production, and memory cell generation. Early in vitro experiments demonstrated that signaling through OX40 on CD4⁺ T cells lead to TH2, but not TH1 development. These results were supported by in vivo studies showing that blocking OX40/OX40L interaction prevented the induction and maintenance of TH2-mediated allergic immune responses. However, blocking OX40/OX40L interaction ameliorates or prevents TH1-mediated diseases. Furthermore, administration of soluble OX40L or gene transfer of OX40L into tumors were shown to strongly enhance anti-tumor immunity in mice. Recent studies also suggest that OX40/OX40L may play a role in promoting CD8 T cell-mediated immune responses. As discussed herein, OX40 signaling blocks the inhibitory function of CD4⁺ CD25⁺ naturally occurring regulatory T cells and the OX40/OX40L pair plays a critical role in the global regulation of peripheral immunity versus tolerance. OX-40 antibodies, OX-40 fusion proteins and methods of using them are disclosed in U.S. Pat. Nos. 7,504,101; 7,758,852; 7,858,765; 7,550,140; 7,960,515; and 9,006,399 and international publications: WO 2003082919; WO 2003068819; WO 2006063067; WO 2007084559; WO 2008051424; WO2012027328; and WO2013028231.

T cell immunoglobulin and mucin domain-containing molecule 3 (TIM3) is an immunoglobulin (Ig) superfamily member, expressed on Th1 cells. TIM3 has been shown to play a role in modulating the immune response of Th1 cells, and reducing inflammation in a number of conditions. TIM3 is also expressed on cancer cells, and on cancer stem cells (CSCs), which are cells that can give rise to additional cancer cells. Antibodies to TIM3 and methods of using in the treatment of disease are described in U.S. Pat. Nos. 7,470,428 and 8, 101, 176.

CTLA-4 is a T cell surface molecule that was originally identified by differential screening of a murine cytolytic T cell cDNA library (Brunet et al., Nature 328:267-270(1987)). CTLA-4 is also a member of the immunoglobulin (Ig) superfamily; CTLA-4 comprises a single extracellular Ig domain. CTLA-4 transcripts have been found in T cell populations having cytotoxic activity, suggesting that CTLA-4 might function in the cytolytic response (Brunet et al., supra; Brunet et al., Immunol. Rev. 103-(21-36 (1988)). Researchers have reported the cloning and mapping of a gene for the human counterpart of CTLA-4 (Dariavach et al., Eur. J. Immunol. 18:1901-1905 (1988)) to the same chromosomal region (2q33-34) as CD28 (Lafage-Pochitaloff et al., Immunogenetics 31:198-201 (1990)). Sequence comparison between this human CTLA-4 DNA and that encoding CD28 proteins reveals significant homology of sequence, with the greatest degree of homology in the juxtamembrane and cytoplasmic regions (Brunet et al., 1988, supra; Dariavach et al., 1988, supra). Yervoy (ipilimumab) is a fully human CTLA-4 antibody marketed by Bristol Myers Squibb. The protein structure of ipilimumab and methods are using are described in U.S. Pat. Nos. 6,984,720 and 7,605,238.

Suitable anti-CTLA4 antibodies for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, ipilimumab, tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO 2001/014424, the antibodies disclosed in PCT Publication No. WO 2004/035607, the antibodies disclosed in U.S. Published Application No. US 2005/0201994, and the antibodies disclosed in granted European Patent No. EP1212422B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. US 2002/0039581 and US 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281.

PD-L1 is a B7 family member that is expressed on many cell types, including APCs and activated T cells (Yamazaki et al. (2002) J. Immunol. 169:5538). PD-L1 binds to both PD-1 and B7-1. Both binding of T-cell-expressed B7-1 by PD-L1 and binding of T-cell-expressed PD-L1 by B7-1 result in T cell inhibition (Butte et al. (2007) Immunity 27:111). There is also evidence that, like other B7 family members, PD-L1 can also provide costimulatory signals to T cells (Subudhi et al. (2004) J. Clin. Invest. 113:694; Tamura et al. (2001) Blood 97:1809). PD-L1 (human PD-L1 cDNA is composed of the base sequence shown by EMBL/GenBank Acc. No. AF233516 and mouse PD-L1 cDNA is composed of the base sequence shown by NM.sub.-021893) that is a ligand of PD-1 is expressed in so-called antigen-presenting cells such as activated monocytes and dendritic cells (Journal of Experimental Medicine (2000), vol. 19, issue 7, p 1027-1034). These cells present interaction molecules that induce a variety of immuno-inductive signals to T lymphocytes, and PD-L1 is one of these molecules that induce the inhibitory signal by PD-1. It has been revealed that PD-L1 ligand stimulation suppressed the activation (cellular proliferation and induction of various cytokine production) of PD-1 expressing T lymphocytes. PD-L1 expression has been confirmed in not only immunocompetent cells but also a certain kind of tumor cell lines (cell lines derived from monocytic leukemia, cell lines derived from mast cells, cell lines derived from hepatic carcinomas, cell lines derived from neuroblasts, and cell lines derived from breast carcinomas) (Nature Immunology (2001), vol. 2, issue 3, p. 261-267). Antibodies to PD-L1 (also referred to as CD274 or B7-H1) and methods for use are disclosed in U.S. Pat. Nos. 7,943,743; 8,383,796; US20130034559, WO2014055897, U.S. Pat. Nos. 8,168,179; and 7,595,048. PD-L1 antibodies are in development as immuno-modulatory agents for the treatment of cancer.

Lymphocyte Activation Gene-3, or LAG-3 (also known as CD223), is a member of the immunoglobulin supergene family and is structurally and genetically related to CD4. LAG-3 is not expressed on resting peripheral blood lymphocytes but is expressed on activated T cells and NK cells. LAG-3 is a membrane protein encoded by a gene located on the distal part of the short arm of chromosome 12, near the CD4 gene, suggesting that the LAG-3 gene may have evolved through gene duplication (Triebel et al. (1990) J. Exp. Med. 171:1393-1405). LAG-3 has been demonstrated to interact with MHC Class II molecules but, unlike CD4, LAG-3 does not interact with the human immunodeficiency virus gp120 protein (Baixeras et al. (1992) J. Exp. Med. 176:327-337). Studies using a soluble LAG-3 immunoglobulin fusion protein (sLAG-3Ig) demonstrated direct and specific binding of LAG-3 to MHC class II on the cell surface (Huard et al. (1996) Eur. J. Immunol. 26:1180-1186).

In in vitro studies of antigen-specific T cell responses, the addition of anti-LAG-3 antibodies led to increased T cell proliferation, higher expression of activation antigens such as CD25, and higher concentrations of cytokines such as interferon-gamma and interleukin-4, supporting a role for the LAG−/MHC class II interaction in down-regulating antigen-dependent stimulation of CD4.sup.+ T lymphocytes (Huard et al. (1994) Eur. J. Immunol. 24:3216-3221). The intra-cytoplasmic region of LAG-3 has been demonstrated to interact with a protein termed LAP, which is thought to be a signal transduction molecule involved in the downregulation of the CD3/TCR activation pathway (Iouzalen et al. (2001) Eur. J. Immunol. 31:2885-2891). Furthermore, CD4.sup.+CD25.sup.+regulatory T cells (T.sub.reg) have been shown to express LAG-3 upon activation and antibodies to LAG-3 inhibit suppression by induced Treg cells, both in vitro and in vivo, suggesting that LAG-3 contributes to the suppressor activity of Treg cells (Huang, C. et al. (2004) Immunity 21:503-513). Still further, LAG-3 has been shown to negatively regulate T cell homeostasis by regulatory T cells in both T cell-dependent and independent mechanisms (Workman, C. J. and Vignali, D. A. (2005) J. Immunol. 174:688-695). Antibodies to LAG-3 and methods of using in treatment of disease are described in US20110150892 and U.S. Pat. No. 6,143,273.

4-1BB (also referred to as CD137, TNFRSF9, etc) is a transmembrane protein of the Tumor Necrosis Factor receptor superfamily (TNFRS). Current understanding of 4-1BB indicates that expression is generally activation dependent and is present in a broad subset of immune cells including activated NK and NKT cells, regulatory T cells, dendritic cells (DC), stimulated mast cells, differentiating myeloid cells, monocytes, neutrophils, and eosinophils (Wang, 2009, Immunological Reviews 229: 192-215). 4-1BB expression has also been demonstrated on tumor vasculature (Broil, 2001, Amer. J Clin. Pathol. 115(4):543-549; Seaman, 2007, Cancer Cell 11: 539-554) and at sites of inflamed or atherosclerotic endothelium (Drenkard, 2007 FASEB J. 21: 456-463; Olofsson, 2008, Circulation 117: 1292-1301). The ligand that stimulates 4-1BB, i.e., 4-1BB Ligand (4-1BBL), is expressed on activated antigen-presenting cells (APCs), myeloid progenitor cells, and hematopoietic stem cells.

Human 4-1BB is a 255 amino acid protein (Accession No. NM.sub.-001561; NP.sub.-001552). The complete human 4-1BB amino acid sequence is provided in SEQ ID NO:68. The protein comprises a signal sequence (amino acid residues 1-17), followed by an extracellular domain (169 amino acids), a transmembrane region (27 amino acids), and an intracellular domain (42 amino acids) (Cheuk A T C et al. 2004 Cancer Gene Therapy 11: 215-226). The receptor is expressed on the cell surface in monomer and dimer forms and likely trimerizes with 4-1BB ligand to signal.

Suitable CD137 (4-1BB) antibodies for use in the methods of the invention, include, without limitation, anti-CD137 antibodies, human anti-CD137 antibodies, mouse anti-CD137 antibodies, mammalian anti-CD137 antibodies, humanized anti-anti-CD137 antibodies, monoclonal anti-CD137 antibodies, polyclonal anti-CD137 antibodies, chimeric anti-CD137 antibodies, anti-4-1BB antibodies, anti-CD137 adnectins, anti-CD137 domain antibodies, single chain anti-CD137 fragments, heavy chain anti-CD137 fragments, light chain anti-CD137 fragments, the antibodies disclosed in U.S. Published Application No. US 2005/0095244, the antibodies disclosed in issued U.S. Pat. No. 7,288,638 (such as 20H4.9-IgG4 [10C7 or BMS-663513] or 20H4.9-IgG1 [BMS-663031]); the antibodies disclosed in issued U.S. Pat. No. 6,887,673 [4E9 or BMS-554271]; the antibodies disclosed in issued U.S. Pat. No. 7,214,493; the antibodies disclosed in issued U.S. Pat. No. 6,303,121; the antibodies disclosed in issued U.S. Pat. No. 6,569,997; the antibodies disclosed in issued U.S. Pat. No. 6,905,685; the antibodies disclosed in issued U.S. Pat. No. 6,355,476; the antibodies disclosed in issued U.S. Pat. No. 6,362,325 [1D8 or BMS-469492; 3H3 or BMS-469497; or 3E1]; the antibodies disclosed in issued U.S. Pat. No. 6,974,863 (such as 53A2); or the antibodies disclosed in issued U.S. Pat. No. 6,210,669 (such as 1D8, 3B8, or 3E1). Additional CD137 agonistic antibodies are described in U.S. Pat. Nos. 5,928,893, 6,303,121 and 6,569,997. Antibodies to 4-1BB(CD137) and methods of using are described, for instance, in U.S. Pat. Nos. 8,137,667; 8,821,867; 8,716,452; and 6,569,997.

CD69 is expressed early and transiently following leukocyte activation after an immune challenge (Cebrian et al., 1988; Hara et al., 1986; Testi et al., 1994) in all hematopoietic subsets except erythrocytes. Although, CD69 is detected in vivo on small subsets of T and B cells in peripheral lymphoid tissues (Sanchez-Mateos et al., 1989), CD69 is persistently expressed in leukocyte infiltrates of several chronic inflammatory diseases such as rheumatoid arthritis, and viral-chronic hepatitis (Garcia-Monzon et al., 1990; Laffon et al., 1991), in leukocytes responsible for graft rejection (Mampaso et al., 1993), in leukocytes involved in the allergic response (Hartnell et al., 1993), in immune cells at the atherosclerotic lesion (Caspar-Bauguil et al., 1998), in tumor infiltrating lymphocytes (Coventry et al., 1996), or upon persistent infection (Zajac et al., 1998). Several reports suggest that CD69 is involved in activation of bone marrow-derived cells (Cebrian et al., 1988; Testi et al., 1994). Nevertheless, nearly normal hematopoietic cell development and T cell maturation occur in CD69.sup.−/− mice under physiological conditions (Lauzurica et al., 2000). Antibodies to CD69 are described in US 20150118237.

As used herein “immuno-modulators” refer to any substance including monoclonal antibodies that effects the immune system. Immuno-modulators can be used as anti-neoplastic agents for the treatment of cancer. For example, immune-modulators include, but are not limited to, anti-CTLA-4 antibodies such as ipilimumab (YERVOY) and anti-PD-1 antibodies (Opdivo/nivolumab and Keytruda/pembrolizumab). Other immuno-modulators include, but are not limited to, OX-40 antibodies, PD-L1 antibodies, LAG3 antibodies, TIM-3 antibodies, 41BB antibodies; ICOS antibodies and GITR antibodies.

T cells require two signals to become fully activated. A first signal, which is antigen-specific, is provided through the T cell receptor (TCR) which interacts with peptide-MHC molecules on the membrane of antigen presenting cells (APC). A second signal, the co-stimulatory signal, is antigen nonspecific and is provided by the interaction between co-stimulatory molecules expressed on the membrane of APC and the T cell. Co-signalling molecules are cell-surface glycoproteins that can direct, modulate and fine-tune TCR signals. On the basis of their functional outcome, co-signalling molecules can be divided into co-stimulators and co-inhibitors, which promote or suppress T-cell activation, respectively. By expression at the appropriate time and location, co-signalling molecules positively and negatively control the priming, growth, differentiation and functional maturation of a T-cell response. As used herein “co-stimulator and/or co-inhibitor receptor” means any receptor expressed on dendritic, APC and/or a Tcell which modulate TCR signal including, but not limited to, B7-CD28-family receptors, Tumor Necrosis Super-Family receptors and Immunoglobulin superfamily. These receptors include, but are not limited to, GITR, B7-H1, B7-DC, B7-H4, CD80, CD86, BTLA, PD-1, OX40, ICOS, CD137, TIM3, and LAG3. As used herein, agents directed to at least one co-stimulatory and/or co-inhibitory receptor include agonists and antagonists to the receptor and/or to the natural ligand of the receptor such as PD-L1, including but not limited to antibodies and or binding fragments thereof.

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor and/or (3) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a co-signalling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, descrease, abrogates the expression of the receptor. Antagonist activity can be measured in vitro by various assays know in the art such as, but not limited to, measurement of an increase or decrease in cell signalling, cell proliferation, immune cell activation markers, cytokine production. Antagonist activity can also be measured in vivo by various assays that measure surrogate end points such as, but not limited to the measurement of T cell proliferation or cytokine production.

As used herein the term “cross competes for binding” refers to any agent such as an antibody that will compete for binding to a target with any of the agents of the present invention. Competition for binding between two antibodies can be tested by various methods known in the art including Flow cytometry, Meso Scale Discovery and ELISA. Binding can be measured directly, meaning two or more binding proteins can be put in contact with a co-signalling receptor and bind may be measured for one or each. Alternatively, binding of molecules or interest can be tested against the binding or natural ligand and quantitatively compared with each other.

The term “binding protein” as used herein refers to antibodies and other protein constructs, such as domains, which are capable of binding to and antigen.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., V_{H}, V_{HH}, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer or an EGF domain.

The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

The term “single variable domain” refers to a folded polypeptide domain comprising sequences characteristic of antibody variable domains. It therefore includes complete antibody variable domains such as V_{H}, V_{HH} and V_{L} and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb™” may be considered the same as a “single variable domain”. A single variable domain may be a human single variable domain, but also includes single variable domains from other species such as rodent nurse shark and Camelid V_{HH} dAbs™. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanized according to standard techniques available in the art, and such domains are considered to be “single variable domains”. As used herein V_{H} includes camelid V_{HH} domains.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

Affinity is the strength of binding of one molecule, e.g. an antigen binding protein of the invention, to another, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE™ analysis). For example, the Biacore™ methods described in Example 5 may be used to measure binding affinity.

Avidity is the sum total of the strength of binding of two molecules to one another at multiple sites, e.g. taking into account the valency of the interaction.

By “isolated” it is intended that the molecule, such as an antigen binding protein or nucleic acid, is removed from the environment in which it may be found in nature. For example, the molecule may be purified away from substances with which it would normally exist in nature. For example, the mass of the molecule in a sample may be 95% of the total mass.

The term “expression vector” as used herein means an isolated nucleic acid which can be used to introduce a nucleic acid of interest into a cell, such as a eukaryotic cell or prokaryotic cell, or a cell free expression system where the nucleic acid sequence of interest is expressed as a peptide chain such as a protein. Such expression vectors may be, for example, cosmids, plasmids, viral sequences, transposons, and linear nucleic acids comprising a nucleic acid of interest. Once the expression vector is introduced into a cell or cell free expression system (e.g., reticulocyte lysate) the protein encoded by the nucleic acid of interest is produced by the transcription/translation machinery. Expression vectors within the scope of the disclosure may provide necessary elements for eukaryotic or prokaryotic expression and include viral promoter driven vectors, such as CMV promoter driven vectors, e.g., pcDNA3.1, pCEP4, and their derivatives, Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g., pET41, lactose promoter driven vectors and arabinose gene promoter driven vectors. Those of ordinary skill in the art will recognize many other suitable expression vectors and expression systems.

The term “recombinant host cell” as used herein means a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells or any derivative thereof. Most preferably, the eukaryotic cell is a HEK293, NS0, SP2/0, or CHO cell. E. coli is an exemplary prokaryotic cell. A recombinant cell according to the disclosure may be generated by transfection, cell fusion, immortalization, or other procedures well known in the art. A nucleic acid sequence of interest, such as an expression vector, transfected into a cell may be extrachromasomal or stably integrated into the chromosome of the cell.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl Acad Sci USA, 86:10029-10032 (1989), Hodgson, et al., Bio/Technology, 9:421 (1991)). A suitable human acceptor antibody may be one selected from a conventional database, e.g., the KABAT™ database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The term “donor antibody” refers to an antibody that contributes the amino acid sequences of its variable regions, CDRs, or other functional fragments or analogs thereof to a first immunoglobulin partner. The donor, therefore, provides the altered immunoglobulin coding region and resulting expressed altered antibody with the antigenic specificity and neutralising activity characteristic of the donor antibody.

The term “acceptor antibody” refers to an antibody that is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

The terms “V_{H}” and “V_{L}” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

In one embodiment, methods are provided for increasing expression of at least one co-stimulatory and/or co-inhibitory receptor on a T cell comprising contacting said T cell with an anti-CTLA4 antibody. In some aspects, the at least one co-stimulatory and/or co-inhibitory receptor is selected from the group of: PD-1, OX40, ICOS, CD137, TIM3, and LAG3. In one embodiment the anti-CTLA4 antibody is ipilimumab.

In one aspect, CD69, PD-1, OX40, and ICOS expression is increased on tumor infiltrating lymphocytes. In another embodiment, the methods of the present invention further comprise increasing levels of TNF-alpha, IL-12p70, IL-13, and IL-5 cytokines.

In one embodiment methods are provided for treating cancer in a human in need thereof comprising administering an anti-CTLA antibody and at least one additional agent directed to at least one co-stimulatory and/or co-inhibitory receptor to said human. In one aspect, the additional agent is directed to at least one co-stimulatory and/or co-inhibitory receptor selected from the group of: PD-1, OX40, ICOS, CD137, TIM3, and LAG3. In one aspect, the anti-CTLA4 antibody is ipilimumab. In one aspect, the agent directed to at least one co-stimulatory and/or co-inhibitory receptor is an agonist antibody directed to OX40. In one aspect, the agent directed to at least one co-stimulatory and/or co-inhibitory receptor is an agonist antibody directed to ICOS. In one aspect, the agent directed to at least one co-stimulatory and/or co-inhibitory receptor is an antagonist antibody directed to TIM3. In one aspect, the agent directed to at least one co-stimulatory and/or co-inhibitory receptor is an antagonist antibody directed to LAG3.

In another embodiment, the methods of the present invention further comprise administering an anti-PD-1 antibody to the human. In one aspect, the anti-PD-1 antibody is selected from pembrolizumab and nivolumab.

In one embodiment, the anti-CTLA-4 antibody and said agent directed to at least one co-stimulatory and/or co-inhibitory receptor are administered to the human simultaneously. In another embodiment, the anti-CTLA-4 antibody and said agent directed to at least one co-stimulatory and/or co-inhibitory receptor are administered to the human sequentially. In one aspect, the methods comprise administering at least one additional neoplastic agent to the human.

In one embodiment, an anti-CTLA4 antibody for use in treating cancer in a human in need thereof is provided, wherein the CTLA4 antibody is administered with at least one additional agent directed to at least one co-stimulatory and/or co-inhibitory receptor in said human. In one aspect, administration of the CTLA4 antibody increases expression of said at least one co-stimulatory and/or co-inhibitory receptor in said human. In one aspect, the additional agent is directed to at least one co-stimulatory and/or co-inhibitory receptor selected from the group of: PD-1, OX40, ICOS, CD137, TIM3, and LAG3. In one aspect, the anti-CTLA4 antibody is ipilimumab. In one aspect, the agent directed to at least one co-stimulatory and/or co-inhibitory receptor is an agonist antibody directed to OX40. In one aspect, the agent directed to at least one co-stimulatory and/or co-inhibitory receptor is an agonist antibody directed to ICOS. In one aspect, the agent directed to at least one co-stimulatory and/or co-inhibitory receptor is an antagonist antibody directed to TIM3. In one aspect, the agent directed to at least one co-stimulatory and/or co-inhibitory receptor is an antagonist antibody directed to LAG3.

In one embodiment, the anti-CTLA4 antibody and said agent directed to at least one co-stimulatory and/or co-inhibitory receptor are administered to the human simultaneously. In another embodiment, the anti-CTLA-4 antibody and said agent directed to at least one co-stimulatory and/or co-inhibitory receptor are administered to the human sequentially. In one aspect, the anti-CTLA4 antibody and said agent directed to at least one co-stimulatory and/or co-inhibitory receptor are administered with at least one additional neoplastic agent to the human.

In another embodiment, the anti-CTLA4 antibody for use in treating cancer in a human in need thereof is administered with an anti-PD-1 antibody to the human. In one aspect, the anti-PD-1 antibody is selected from pembrolizumab and nivolumab.

In one embodiment, an anti-CTLA4 antibody and at least one additional agent directed to at least one co-stimulatory and/or co-inhibitory receptor in said human for simultaneous or sequential use in treating cancer in a human in need thereof are provided. In one aspect, administration of the CTLA4 antibody increases expression of said at least one co-stimulatory and/or co-inhibitory receptor in said human. In one aspect, the additional agent is directed to at least one co-stimulatory and/or co-inhibitory receptor selected from the group of: PD-1, OX40, ICOS, CD137, TIM3, and LAG3. In one aspect, the anti-CTLA4 antibody is ipilimumab. In one aspect, the agent directed to at least one co-stimulatory and/or co-inhibitory receptor is an agonist antibody directed to OX40. In one aspect, the agent directed to at least one co-stimulatory and/or co-inhibitory receptor is an agonist antibody directed to ICOS. In one aspect, the agent directed to at least one co-stimulatory and/or co-inhibitory receptor is an antagonist antibody directed to TIM3. In one aspect, the agent directed to at least one co-stimulatory and/or co-inhibitory receptor is an antagonist antibody directed to LAG3.

In one embodiment, the anti-CTLA4 antibody and said agent directed to at least one co-stimulatory and/or co-inhibitory receptor are for simultaneous use. In one embodiment, the anti-CTLA4 antibody and said agent directed to at least one co-stimulatory and/or co-inhibitory receptor are for sequential use. In one aspect, the anti-CTLA4 antibody and said agent directed to at least one co-stimulatory and/or co-inhibitory receptor are administered with at least one additional neoplastic agent to the human.

In another embodiment, the anti-CTLA4 antibody and said agent directed to at least one co-stimulatory and/or co-inhibitory receptor for simultaneous or sequential use in treating cancer in a human in need thereof is administered with an anti-PD-1 antibody to the human. In one aspect, the anti-PD-1 antibody is selected from pembrolizumab and nivolumab.

In one aspect the cancer is selected from head and neck cancer, breast cancer, lung cancer, colon cancer, ovarian cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

In one aspect, the methods of the present invention further comprise administering at least one neo-plastic agent to said human.

In one aspect the human has a solid tumor. In one aspect the tumor is selected from head and neck cancer, gastric cancer, melanoma, renal cell carcinoma (RCC), esophageal cancer, non-small cell lung carcinoma, prostate cancer, colorectal cancer, ovarian cancer and pancreatic cancer. In another aspect the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent Bcell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphomas(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present methods are anti-neoplastic agents including any chemotherapeutic agents, immuno-modulatory agents or immune-modulators and immunostimulatory adjuvants.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994, lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)—2,3-dihydroxybutanedioate (1:2)(salt)], commercially available as an injectable solution of vinorelbine tartrate (NAVELBINE®), is a semisynthetic vinca alkaloID Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)—O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-ibis(2-chloroethyl)aminol-L-phenylalanine, is commercially available as an injectable solution or tablets as ALKERAN®. Melphalan is indicated for the palliative treatment of multiple myeloma and non-reselectable epithelial carcinoma of the ovary. Bone marrow suppression is the most common dose limiting side effect of melphalan.

Chlorambucil, 4-[bis(2-chloroethyl)aminoThenzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′, 2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia CLL; a type of cancer of the white blood cells) in adults who are refractory to treatment with fludarabine (Fludara) and alemtuzumab Campath).

Trastuzumab (HEREPTIN®) is a humanized monoclonal antibody that binds to the HER2 receptor. It original indication is HER2 positive breast cancer.

Cetuximab (ERBITUX®) is a chimeric mouse human antibody that inhibits epidermal growth factor receptor (EGFR).

mTOR inhibitors include but are not limited to rapamycin (FK506) and rapalogs, RAD001 or everolimus (Afinitor), CCI-779 or temsirolimus, AP23573, AZD8055, WYE-354, WYE-600, WYE-687 and Pp121.

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl) ethenyl] benzoic acID Bexarotene is used to treat cutaneous T-cell lymphoma CTCL, a type of skin cancer) in people whose disease could not be treated successfully with at least one other medication.

Sorafenib marketed as Nexavar® is in a class of medications called multikinase inhibitors. Its chemical name is 4-[4-[[4-chloro-3-(trifluoromethyl)phenyl]carbamoylamino] phenoxy]-N-methyl-pyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

Examples of erbB inhibitors include lapatinib, erlotinib, and gefitinib. Lapatinib, N-(3-chloro-4-{[(3-fluorophenyl)methyl]oxy}phenyl)-6-[5-({[2-(methylsulfonyl)ethyl]amino}methyl)-2-furanyl]-4-quinazolinamine (represented by formula II, as illustrated), is a potent, oral, small-molecule, dual inhibitor of erbB-1 and erbB-2 (EGFR and HER2) tyrosine kinases that is approved in combination with capecitabine for the treatment of HER2-positive metastatic breast cancer.

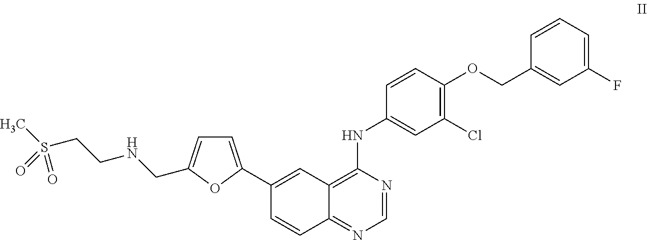 [SMILES: CS(=O)(=O)CCNCc1ccc(-c2ccc3ncnc(Nc4ccc(OCc5cccc(F)c5)c(Cl)c4)c3c2)o1]

The free base, HCl salts, and ditosylate salts of the compound of formula (II) may be prepared according to the procedures disclosed in WO 99/35146, published Jul. 15, 1999; and WO 02/02552 published Jan. 10, 2002.

Erlotinib, N-(3-ethynylphenyl)-6,7-bis{[2-(methyloxy)ethyl]oxy}-4-quinazolinamine Commercially available under the tradename Tarceva) is represented by formula III, as illustrated:

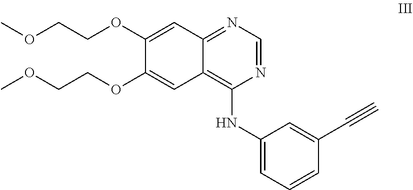 [SMILES: C#Cc1cccc(Nc2ncnc3cc(OCCOC)c(OCCOC)cc23)c1]

The free base and HCl salt of erlotinib may be prepared, for example, according to U.S. Pat. No. 5,747,498, Example 20.

Gefitinib, 4-quinazolinamine,N-(3-chloro-4-fluorophenyl)-7-methoxy-6-[3-4-morpholin)propoxy] is represented by formula IV, as illustrated:

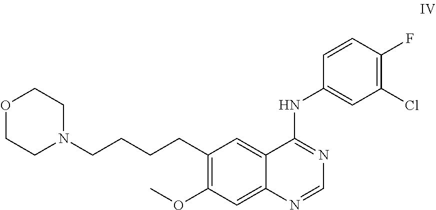 [SMILES: COc1cc2ncnc(Nc3ccc(F)c(Cl)c3)c2cc1CCCCN1CCOCC1]

Gefitinib, which is commercially available under the trade name IRESSA® (Astra-Zenenca) is an erbB-1 inhibitor that is indicated as monotherapy for the treatment of patients with locally advanced or metastatic non-small-cell lung cancer after failure of both platinum-based and docetaxel chemotherapies. The free base, HCl salts, and diHCl salts of gefitinib may be prepared according to the procedures of International Patent Application No. PCT/GB96/00961, filed Apr. 23, 1996, and published as WO 96/33980 on Oct. 31, 1996.

Also of interest, is the camptothecin derivative of formula A following, currently under development, including the racemic mixture (R,S) form as well as the R and S enantiomers:

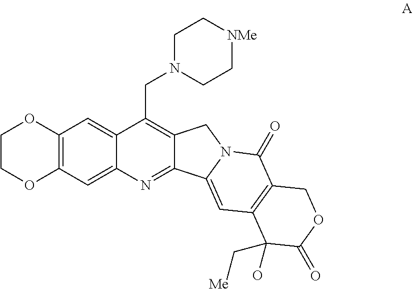 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal transduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor—I (IGFI) receptor, macrophage colony stimulating factor Cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994 New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4 292-8; Ashby, M. N. (1998), Current Opinion in Lipidology. 9 (2) 99-102; and Bennett, C. F. and Cowsert, L. M. BioChim. Biophys. Acta, (1999) 1489(1):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4, 269-286); Herceptin erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124.

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994, Antisense Res. Dev. 4: 71-79.

Trastuzumab (HEREPTIN®) is a humanized monoclonal antibody that binds to the HER2 receptor. It original indication is HER2 positive breast cancer.

Trastuzumab emtansine (trade name Kadcyla) is anantibody-drug conjugate consisting of the monoclonal antibody trastuzumab (Herceptin) linked to the cytotoxic agent mertansine (DM1). Trastuzumab alone stops growth of cancer cells by binding to the HER2/neu receptor, whereas mertansine enters cells and destroys them by binding to tubulin. Because the monoclonal antibody targets HER2, and HER2 is only over-expressed in cancer cells, the conjugate delivers the toxin specifically to tumor cells. The conjugate is abbreviated T-DM1.

Cetuximab (ERBITUX®) is a chimeric mouse human antibody that inhibits epidermal growth factor receptor (EGFR).

Pertuzumab (also called 2C4, trade name Omnitarg) is a monoclonal antibody. The first of its class in a line of agents called “HER dimerization inhibitors”. By binding to HER2, it inhibits the dimerization of HER2 with other HER receptors, which is hypothesized to result in slowed tumor growth. Pertuzumab is described in WO01/00245 published Jan. 4, 2001.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

Cell cycle signalling inhibitors inhibit molecules involved in the control of the cell cycle. A family of protein kinases called cyclin dependent kinases CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

As used herein “immunostimulatory agent” refers to any agent that can stimulate the immune system. As used herein immunostimulatory agents include, but are not limited to, vaccine adjuvants.

### EXAMPLES

The following examples illustrate various non-limiting aspects of this invention.

### Example 1

The purpose of these studies was to determine the effect of Ipilimumab treatment on T cell expansion, activation, cytokine production, tumor infiltration, and expression level of co-stimulatory and co-inhibitory receptors in a tumor bearing humanized NSG mouse model. Antibody therapies for immune modulation are proving to be effective in the oncology setting, as demonstrated by anti-CTLA-4 Ipilimumab and anti-PD-1 Pembrolizumab clinical activity. In order to assess efficacy of new therapies in the context of immune activation and tumor response, there is a need for suitable preclinical in vivo models. One approach to study immune cell function is to inject human peripheral blood mononuclear cells (PBMCs) into adult immunodeficient NSG (NOD/SCID/IL-2Rγnull) mice. This model, known as Hu-PBMC NSG, induces a Graft-versus-Host Disease (GvHD) state and has been used to study effector and memory T cell activity. Here we utilized the Hu-PBMC NSG model implanted with human cancer cell lines to investigate the effect of Ipilimumab on T cells and tumor growth in vivo. All studies were conducted in accordance with the GSK Policy on the Care, Welfare and Treatment of Laboratory Animals and were reviewed the Institutional Animal Care and Use Committee at GSK. The human biological samples were sourced ethically and their research use was in accord with the terms of the informed consents.

### Procedure:

### Preparation of Cell Lines:

Human A2058 and 786-O cancer cell lines were propagated according to the American Type Culture Collection (ATCC) protocol.

### Materials:

A2058 human melanoma cell line: ATCC, catalog number CRL-11147, lot number 59349362.
786-O human Renal Adenocarcinoma: ATCC, catalog number CRL-1932, lot number 60888404
DPBS: ATCC, catalog number 30-2200, lot number 63357436
RPMI1640: ATCC, catalog number 30-2001, lot number 62027197
Dulbecco's Modified Eagle's Medium: ATCC, catalog number 30-2002, lot number 62596471
Fetal Bovine Serum: Sigma-Aldrich, catalog number 12176c-1000 mL, lot number 13G180R0H1
0.25% (w/v) Trypsin-0.53 mM EDTA: ATCC, catalog number 30-2102, lot number 62420300
Antibiotic-Antimycotic (100×): Life Technologies, catalog number 15240-062
CryoStor®CS10 freezing media: Biolife solutions, catalog number 210102
T175 cell culture flask: Greiner bio-one, catalog number 661175
T75 cell culture flask: Greiner bio-one, catalog number 658175

### Medium:

A2058 complete growth medium: Dulbecco's Modified Eagle's Medium+10% FBS+1×antibiotic-antimycotic.
786-O complete growth medium: RPMI-1640 Medium+10% FBS+1×antibiotic-antimycotic.
   Culture conditions: Atmosphere: Air, 95%; 5% carbon dioxide (CO2); Temperature: 37° C.

### Cell Preparation:

Pre-warm complete medium at 37° C.
Thaw frozen cells quickly in a 37° C. water bath. Wipe the outside of the tube with 70% ethanol and transfer cells to 15 ml tube containing prewarmed complete medium.
Centrifuge at 1200 rpm for 5 minutes to pellet cells.
Add cells to a T75 tissue culture flask with prewarmed complete medium and incubate at 37° C.

### Subcultureing of the Cells:

Volumes indicated are for a 75 cm² tissue culture flask. For T175 cm² flask, adjust volumes proportionally).
Remove and discard culture medium form the tissue culture flask
Briefly rinse the adherent cell layer with DPBS to remove residual serum. Add 2.0 to 3.0 mL of Trypsin-EDTA solution to the flask and observe until cells begin to detach. To avoid clumping do not agitate cells by hitting or shaking the flask. The flask may be incubated at 37° C. to facilitate dispersal.
Add 10 mL of complete growth medium to the flask and aspirate cells by gently pipetting.
Centrifuge at 1200 rpm for 5 minutes to pellet the cells. Aspirate medium, and resuspend the cell pellet in 10 ml of complete growth medium.
Add appropriate aliquots of the cell suspension to new culture vessels with fresh medium. Incubate tissue cultures at 37° C.
Subculture the cells every 2 to 3 days when the cell monolayer becomes approximately 80% confluent.

### Cryopreservation:

Freeze aliquots of the cells in CS10 (5 million cells per vial) according to the vendor's protocol.
Wash the cell monolayer with 1×DPBS and add 3 ml 1×Trypsin for 2-3 minutes.
Once the cells have dispersed, add complete growth media and collect the cell suspension in a sterile conical centrifuge tube.
Centrifuge the cells at 1200 rpm for 5 minutes to pellet the cells.
Resuspend the cell pellet in CS10 freeze medium at a concentration of 5 million cells per mL.
Incubate the cells at 4° C. for 5-10 minutes, and then transfer to −80 for 2 hours before transferring to liquid nitrogen for long term storage.

### Preparation of Tumor Cells for Mouse Inoculation:

Wash cell monolayer with 1×DPBS and add 3 mL of 1×Trypsin for 2-3 minutes.
Once the cells have dispersed, add complete growth media and collect the cell suspension in a sterile conical centrifuge tube.
Centrifuge the cells at 1200 rpm for 5 minutes to pellet the cells.
Resuspend and wash the cells with 1×DPBS, and centrifuge at 1200 rpm for 5 minutes to pellet the cells.
Resuspend the cells in ice-cold sterile PBS at concentrations appropriate for mouse inoculations (A2058: 25 million cells/mL. 100 uL containing 2.5 million cells is inoculated per mouse. 786-o: 10 million cells/mL 100 uL containing 1 million cells is inoculated per mouse.) Maintain the cells on ice.

NSG Mouse Tumor Cell Line Inoculation

Materials:
Mice: NOD.Cg-Prkdcscid Il2rgtm1Wjl/SzJ. The Jackson Laboratory Stock: 005557 Female Age: 6 weeks.
1 mL Tuberculin Syringes with Attached Needle 25 G 5/8: Becton Dickinson, catalog number 305554
PDI™ Alcohol Prep Pads: Professional Disposables, catalog number B339
PDI™ Povidone-Iodine Prep Pad: Professional Disposables, catalog number B40600

### Preparation of Mice

Mice are 6 weeks old.
Shave the right hind flank of the mice for tumor cell inoculation.

### Preparation for the Injection

Clean and sterilize the inoculation area of the mice with an ethanol and/or iodine solution.
Use a 1-cc syringe and a 25-gauge needle with a 100 ul cell stock solution.
Inject 100 ul of cells subcutaneously (s.c.) into the right hind flank of each mouse.

### Tumor Growth Assessment

Measure tumor size and body weight every 2-3 days.
The tumor size is measured with a digital caliper, and the volume is determined by the formula: Tumor volume (cm3)=(length)×(width)/2.

Human PBMCs can be injected approximately 1 week after tumor cell inoculation when the tumors reach an average volume of approximately 100 mm³.

### Human PBMC Intravenous Administration

### Materials:

Fresh Human PBMCs: Allcells, catalog number C-PB102-2B8.
1 mL Tuberculin Syringes with Attached Needle 25 G 5/8: Becton Dickinson, catalog number 305554.
PDI™ Alcohol Prep Pads: Professional Disposables, catalog number B339.
PDI™ Povidone-Iodine Prep Pad: Professional Disposables, catalog number B40600.
Gauze sponges: Covidien, catalog number 441211.
Mouse Tail Illuminator Restrainer: Braintree scientific, catalog number MTI STD.

### PBMC Preparation

Freshly isolated human peripheral blood mononuclear cells (PBMCs) from Allcells are shipped overnight for next day inoculation.
Centrifuge the cells at 1400 rpm for 5 minutes to pellet the cells.
Wash cells with a 1×DPBS solution, and centrifuge at 1400 rpm for 5 minutes to pellet the cells.
Resuspend the cells in ice-cold PBS at a concentration of 40 million cells/mL.
Keep the cells on ice.
   Mouse Tail Vein injection
Warm the mice with an incandescent lamp for 5 minutes.
Restrain the mice with a tail illuminator restrainer.
Rotate the tail slightly to visualize the vein.
Clean and sterilize the injection site with an ethanol and/or iodine solution.
Use 1-cc syringe and a 25-gauge needle to inject cells.
Insert the needle into the vein at a slight angle. Inject 500 uL of the 40 million cells/mL cell stock.
Remove the needle and apply gentle compression with a gauze sponge until any bleeding has stopped.
Return the mice to their cage and observe for 5-10 minutes to confirm, no tail vein bleeding occurs.

### Therapeutic Antibody Administration

Two to Three days post human PBMC injection, the mice are administrated with antibodies by intraperitoneal injection. The dose schedule for the human IgG1 isotype control and Ipilimumab (Yervoy) antibodies are dosed at 3 mg/kg, twice a week for 3 weeks.

### Materials:

Fully human IgG1 isotype control: Eureka therapeutics, catalog number ET-901 (preclinical grade) Lot number 15-726.
Ipilimumab (Yervoy): Bristol-Myers Squibb NDC 0003-2327-11, lot number 921873.
Mouse IgG2a K Isotype Control Functional Grade: Ebioscience, catalog number 16-4724, lot number E06664-1632.
Anti-Human CD3 Functional Grade Purified (OKT3): Ebioscience, catalog number 16-0037-85, lot #E06305-167.
½ mL BD™ Tuberculin syringe with 27 G: Becton Dickinson, Catalog number 305620. Intraperitoneal injection:
100 ul of the antibody to be administered is drawn into a syringe.
Line the bevel of the needle with the numbers on the syringe.
Sufficiently restrain the animal with the non-dominant hand.
The entry point for the needle is determined as follows: draw an imaginary line across the abdomen just above the knees. The needle will be inserted along this line on the animal's right side and close to the midline. With female mice the point of entry is cranial to and slightly medial of the last nipple.
Tilt the mouse with its head slightly downward so the head is lower than the hind end.
Insert the needle into the abdomen at a 30-degree angle.
The shaft of the needle should enter to a depth of approximately half a centimeter.
After injection, withdraw the needle and return the mouse to its cage.

### Blood and Tumor Sampling

### Materials:

Microvette CB300 (Serum): Braintree Scientific, Catalog number MV-CB300 16440
Microvette CB300 (Hematology/Potassium EDTA): Braintree Scientific, Catalog number MV-CB300 16444

### Blood:

Mice are tail vein bled once a week.
30 ul of blood is collected in Microvette CB300 (Hematology/Potassium EDTA) for flow cytometry analysis.
Another 30 ul of blood is collected in serum Microvette CB300 and incubated for 2 h at room temperature to allow clotting, followed by centrifugation at 2000×g in order to collect serum. Serum is stored at −20 until further analysis.

### Tumor:

Mice are euthanized when tumor size is over 2000 mm³. Tumors are collected and processed in the following procedure below.

### Tumor Dissociation and Tumor Infiltrating Lymphocytes Isolation

Tumor tissues are dissociated into single-cell suspensions by combining mechanical dissociation with enzymatic degradation of the extracellular matrix, which maintains the structural integrity of tissues using human Tumor dissociation kit from Miltenyi Biotec. The Tumor Dissociation Kit has been developed for the gentle, rapid, and effective generation of single-cell suspensions from primary human tumor tissue or xenografts. It is optimized for a high yield of tumor cells and tumor infiltrating lymphocytes (TILs), while preserving cell surface epitopes.

### Materials:

Tumor dissociation kit (Human): Miltenyi Biotec, Catalog number 130-095-929, lot #5150407521.
RoboSep™ Buffer: Stemcell Technology, catalog number 422302.
Ficoll-Paque plus: GE Healthcare Life Science, Catalog number 17-1440-03
Disposable Scapel: Miltex, Catalog number 4-421
50 ml conical tube: Falcon, Catalog number 352070

### Reagent Preparation

Prepare Solution 1 by reconstitution of the lyophilized powder in each vial with 3 mL of RPMI 1640. Prepare aliquots of appropriate volume and avoid repeated freeze-thaw-cycles. Store aliquots at −20° C.
Prepare Solution 2 by reconstitution of the lyophilized powder in the vial with 2.7 mL RPMI 1640. Prepare aliquots of appropriate volume and avoid repeated freeze-thaw-cycles. Store aliquots at −20° C.
Prepare Solution 3 by reconstitution of the lyophilized powder in the vial with 1 mL Reconstitution Buffer for Solution 3 supplied with the kit. Do not vortex. Prepare aliquots of appropriate volume and avoid repeated freeze-thaw-cycles. Store aliquots at −20° C.
Prepare enzyme mix by adding 4.7 mL of RPMI 1640, 200 μL of Solution 1, 100 μL of Solution 2, and 25 μL of solution 3 into a 50 ml conical tube.
Cut the tumor into small pieces of 2-4 mm. Remove fat, fibrous and necrotic areas from the tumor sample.
Transfer the tissue pieces into the 50 ml tube containing the enzyme mix.
Tightly close the tube and incubate the sample for 30 minutes at 37° C. under continuous rotation.
Apply the cell suspension to an appropriate cell strainer placed on a 50 mL tube.
Wash cell strainer with RoboSep™ Buffer
Centrifuge the cell suspension at 300×g for 5 minutes. Aspirate supernatant completely.
Resuspend cells in 30 ml RoboSep™ Buffer, underlay 15 ml of Ficoll-Paque plus to each tube gently.
Centrifuge at room temperature (15-25° C.) for 30 minutes at 1400 rpm with brake off.
Remove and discard upper layer without disturbing Ficoll interface.
Remove and transfer mononuclear cell layer at the interface without disturbing erythrocyte/granulocyte/Tumor cell pellet to a new 50 ml conical tube.
Top the tube with RoboSep™ Buffer. Centrifuge cell suspension at 1400 rpm for 5 minutes. Aspirate supernatant completely.
Resuspend cells in 1 ml Flow staining buffer (BSA). Resuspend cells are ready for flow cytometry analysis.

### Flow Cytometry of Whole Mouse Blood

Whole blood is stained for flow cytometry before RBC lysis.
Aliquot 10 μL of whole blood to each well.
Pre-incubate the cells with 5 μL of Human TruStain FcX™ and TruStain fcX™ (anti-mouse CD16/32) per well for 10-20 minutes on ice prior to staining.
Combine the recommended quantity of each primary antibody in an appropriate volume of Flow Cytometry Staining Buffer so that the final volume of antibody mix is 100 μL.
Add to cells and pulse vortex gently to mix. Incubate for 30 minutes on ice. Protect from light.
Transfer cells to a 2 ml deep well 96 well block, without washing cells, add 1 mL of freshly prepared 1×RBC lysing solution prepared as indicated above and pulse vortex briefly. Incubate for 20 minutes at room temperature. Protect from light. Note: Do not exceed 20 minutes of incubation with RBC Lysis Buffer.
Centrifuge samples at 1400 rpm for 5 minutes at room temperature, discard supernatant
Wash the cells twice with 2 mL of Flow Cytometry Staining Buffer. Pellet the cells by centrifugation at 1400 rpm for 5 minutes at room temperature
Resuspend stained cells in 300 ul of Flow cytometry staining buffer.
Transfer cells to 96 well round bottom plate, acquire data on a flow cytometer

### Flow Cytometry of Tumor Infiltrating Lymphocytes

Add cell suspension (from 2×10⁵-10⁷) to each well of 96 well round bottom plate
Block non-specific Fc-mediated interactions. Pre-incubate the cells with 5 μL of Human TruStain FcX™ and TruStain fcX™ (anti-mouse CD16/32) per well for 10-20 minutes on ice prior to staining
Combine the recommended quantity of each primary antibody in an appropriate volume of Flow Cytometry Staining Buffer so that the final volume of antibody mix is 100 μL.
Add to cells and pulse vortex gently to mix. Incubate for 30 minutes on ice. Protect from light.
Centrifuge samples at 1400 rpm for 5 minutes at room temperature, discard supernatant
Wash the cells twice with 200 ul of Flow Cytometry Staining Buffer. Pellet the cells by centrifugation at 1400 rpm for 5 minutes at room temperature
Resuspend stained cells in 300 ul of Flow Cytometry Staining Buffer. Acquire data on a flow cytometer

### MSD Cytokines Analysis

### Materials:

V-plex MSD kit: Meso Scale Discovery: Catalog number N05JA-1, Lot #20045286
MSD Sector Imager 2400 plate reader: Meso Scale Discovery
Discovery Workbench 3.0 software: Meso Scale Discovery

Meso Scale Discovery multiplex assay kits allow quantitation of multiple analytes in the same sample, which preserves precious specimens and provides significant savings in costs and lab time. To measure cytokines, the customized human cytokines kit is ordered from MSD which provides assay-specific components for the quantitative determination of IFN-γ, IL-2, IL-4, IL-5,IL-6, IL-10, IL-12p70, IL-13, and TNF-α. All reagents were provided with the kit.
The standards were reconstituted and serum was 1:10 diluted into in the assay-diluent provided.
25 μl of samples and standards were added per well and incubated for 2 h at room temperature on an plate shaker.
At the end of the incubation, the wells were washed, detection antibody was added and the plates were incubated and washed again according to the manufacturers' instructions.
150 μl of MSD Reading Buffer was added to the wells and the plates were analyzed on the MSD Sector Imager 2400 plate reader.
The data were measured as electro-chemiluminescence signal detected by photo-detectors and analyzed using Discovery Workbench 3.0 software.
A best-fit curve was generated for each analyte using the standards and the concentration of each sample calculated.

### Conclusion:

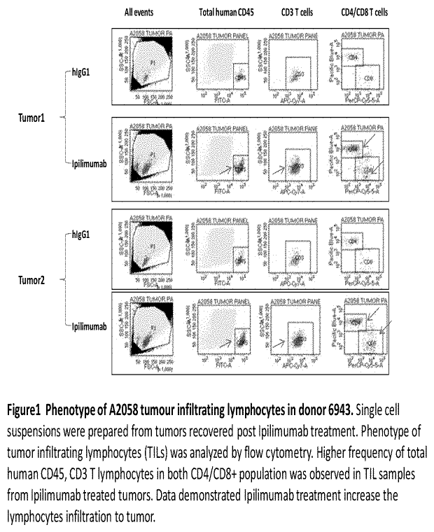

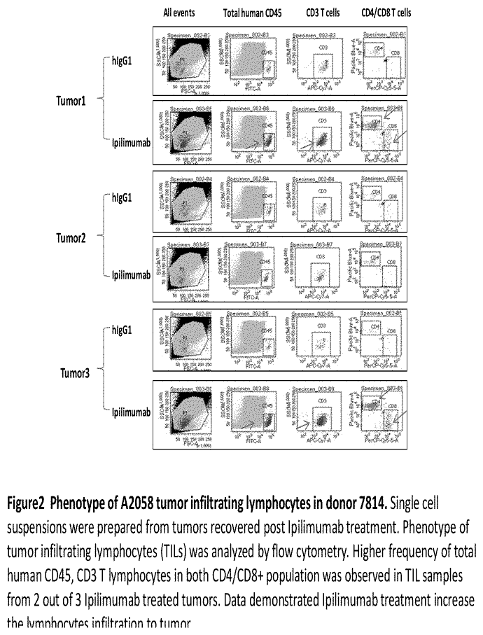

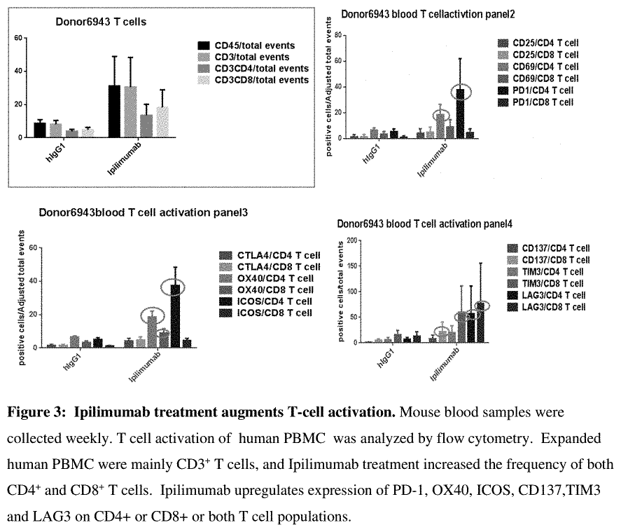

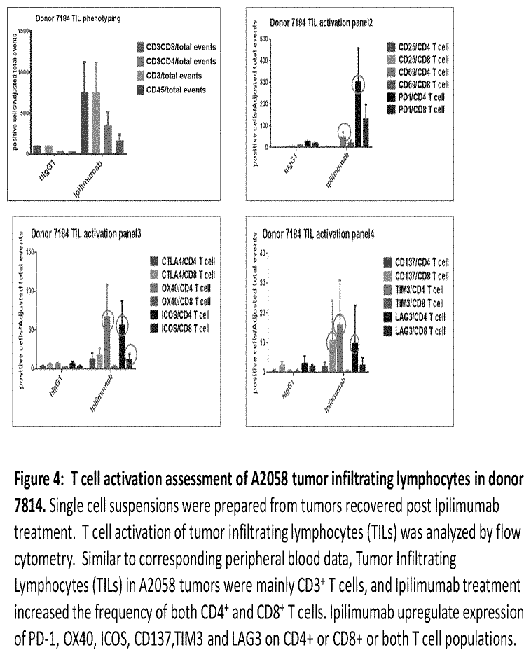

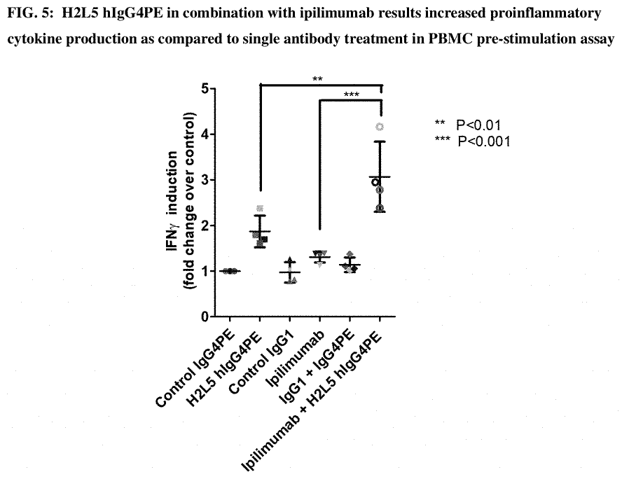

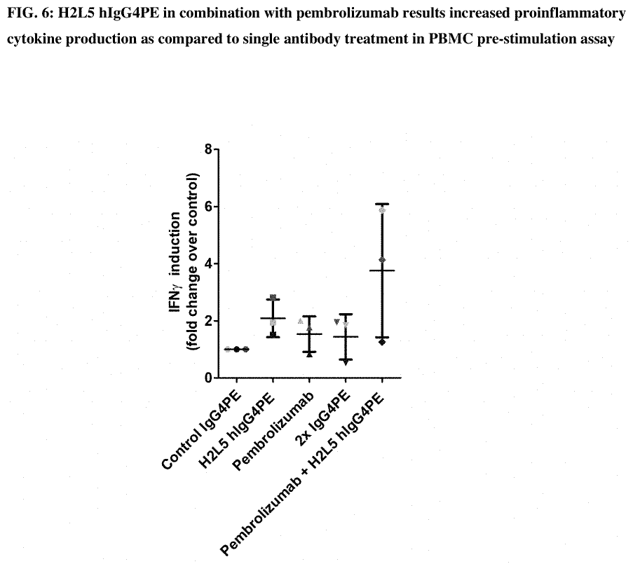

The purpose of these studies was to determine the effect of Ipilimumab treatment on T cell expansion, activation, cytokine production, tumor infiltration, and expression level of co-stimulatory and co-inhibitory receptors in a tumor bearing humanized NSG mouse model. Antibody therapies for immune modulation are proving to be effective in the oncology setting, as demonstrated by anti-CTLA-4 Ipilimumab and anti-PD-1 Pembrolizumab clinical activity. In order to assess efficacy of new therapies in the context of immune activation and tumor response, there is a need for suitable preclinical in vivo models. One approach to study immune cell function is to inject human peripheral blood mononuclear cells (PBMCs) into adult immunodeficient NSG (NOD/SCID/IL-2Rγnull) mice. This model, known as Hu-PBMC NSG, induces a Graft-versus-Host Disease (GvHD) state and has been used to study effector and memory T cell activity. Here we utilized the Hu-PBMC NSG model implanted with human cancer cell lines to investigate the effect of Ipilimumab on T cells and tumor growth in vivo. To accomplish this we confirmed human PBMC engraftment in NSG mice, validated tumor growth of human A2058 melanoma and 786-O renal adenocarcinoma cell lines, and dosed Ipilimumab in tumor bearing NSG mice with different human PBMC donors. Naïve NSG mice were intravenously injected with 20×10⁶ human PBMCs and the kinetics of human cell engraftment was monitored weekly. For studies including Ipilimumab dosing, mice were inoculated with a subcutaneous injection of 2.5×10⁶ A2058 or 1×10⁶ 786-O tumor cells. As tumor size reached 100 mm³, mice were randomized and injected with 20×10⁶ human PBMCs. Two days later, mice were intraperitoneally treated with Ipilimumab or human IgG1 isotype control twice weekly for 6 doses total. Tumor growth and body weight was evaluated over time. Peripheral blood was collected weekly for analysis of T cell activation, receptor expression levels, and human cytokine production until mice developed GvHD or tumor volume reached 2,000 mm³. Select tumors were also harvested for tumor infiltrating lymphocyte (TIL) analysis by flow cytometry. Our studies showed NSG mice demonstrated similar human CD45⁺ cell engraftment in blood with various PBMC donors. The frequency of circulating human CD45⁺ lymphocytes in mouse peripheral blood increased to 50% or greater until week 4. 95% of the CD45⁺ cells were CD3⁺ T cells, containing both CD4⁺ and CD8⁺ subsets. Signs of GvHD were observed at week 4, and serum cytokine analysis showed high levels of GvHD markers e.g. IL5, IL10, and TNF-alpha. Ipilimumab treatment delayed tumor growth, increased the expansion of human CD45⁺ cells, and induced higher levels of TNF-alpha, IL-12p70, IL-13, and IL-5 cytokines compared to isotype control. Ipilimumab also increased the surface expression level of CD69, PD-1, OX40, ICOS, CD137, TIM3, and LAG3 on circulating T cells, and increased the number of A2058 tumor infiltrating lymphocytes (FIGS. 1 and 2). TIL analysis showed that compared to isotype control, Ipilimumab increased expression of CD69, PD-1, OX40, and ICOS on tumor infiltrating lymphocytes (FIG. 4). Ipilimumab upregulates expression of PD-1, OX40, ICOS, CD137, TIM3 and LAG3 on CD4⁺ or CD8⁺ or both T cell populations taken from peripheral blood and tumor infiltrating lymphocytes (FIGS. 3 and 4). In conclusion, Ipilimumab treatment increased T cell expansion, activation, expression of co-stimulatory and co-inhibitory receptors, and cytokine production in this tumor-bearing Hu-PBMC NSG model. It also increased the number of tumor infiltrating lymphocytes with a corresponding tumor growth delay. Based on Ipilimumab activity, this model can be utilized to assess pre-clinical efficacy of novel immunotherapies.

### Example 2: Functional Effects of Soluble H2L5 hIgG4PE Alone and in Combination with Anti-PD1 and Anti-CTLA-4 Antibody in Human PBMC Assay

### Experimental Preparation(s)

### Isolation of Primary Human PBMC

Fresh blood was obtained from GSK Health Center blood donors and was diluted 1:1 with phenol red free-10% RPMI1640 media. Diluted blood was layered on top of the density medium in a Uni-Sep Max 50 ml conical tube and centrifuge at 400×g for 20 minutes at room temperature with BREAK OFF. The resulted white mononuclear layer (buffy coat) was carefully extracted into a new 50 mL conical tube through a 100 uM cell strainer. An equal volume of Phenol red free-10% RPMI1640 media was added to the buffy coat and centrifuged at 300×g for 10 minutes at room temperature. The cell pellet was resuspended in 10 ml of red blood cell lysis solution (Sigma Aldrich) and incubated for 5 minutes at room temperature. Cells were washed once with media and centrifuged as previously described. Volume was brought to 40 ml with Phenol red free-10% RPMI1640 media and cells were counted using Vicell cell counter and viability analyzer (Beckman Coulter).

Induction of Monocyte-Derived Immature Dendritic Cells (iDC)

Human monocytes were isolated using the plastic adherence method. Briefly, 20 million freshly isolated PBMC were cultured in a T-75 tissue culture flask in AIM-V media (Thermo Fisher) for 3 hours. Cells that do not bind to plastic were washed off. The adherent monocytes were cultured in a 37° C. 5% CO₂ incubator in AIM-V media supplemented with 1000U/ml of human GM-CSF (Calt #300-03, PeproTech) and 500 Um′ of human IL-4 (cat #200-04). After 7-10 days, the iDC cells were collected for co-culturing with T cells from a different donor in the allogeneic Mixed Lymphocyte Reaction assays.

Isolation of Primary Human T Cells Directly from Blood

Human T cells were isolated directly from fresh human blood using a human T cell enrichment cocktail (Stem Cell Technologies). The RosetteSep Human T Cell Enrichment Cocktail (50 μL/mL) was added to whole blood and mixed well. After 20 minutes of incubation at room temperature, an equal volume of PBS+2% FBS was added with gentle mixing. The diluted sample was layered on top of the density medium and centrifuged for 20 minutes at 1200×g at room temperature with the brake off. The enriched cells from the density medium: plasma interface were carefully poured into a new conical tube. Next, the red blood cells were lysed with Red Blood Cell Lying Buffer (Sigma Aldrich) and the enriched cells were washed with PBS+2% FBS twice. The T cells were then resuspended in 40 ml of PBS+2% FBS and counted with a Vi-Cell cell counter.

### Experimental Protocols

### Human PBMC Pre-Stimulation Assay

Freshly isolated human PBMCs were pre-stimulated with CD3/CD28 T cell expander DynaBeads at a bead to cell ratio of 1:20 in a T-75 tissue culture flask in AIM-V medium supplemented with 100 ng/ml of MCSF and 100 IU/ml of IL-2 (PeproTech) at 37° C. After 48 hours, the pre-stimulation beads were magnetically removed and cells were washed, counted and re-stimulated with anti-CD3 DynaBeads and therapeutic antibodies in AIM-V medium supplemented with 100 IU/ml of IL-2 (PeproTech) in 96-well non-tissue culture treated round bottom plate. The seeding density was 100k cells per 100 ul of medium per well. After incubating at 37° C. for 3.5 days, cell culture supernatants were collected for multiplex cytokine measurement by MSD.

### Human MLR Activation Assay

Monocyte-derived iDCs from a healthy human volunteer were mixed at a 1:10 ratio (iDC:T) with freshly isolated human T cells from a different donor and pre-incubated at 37° C. in AIM-V media in the presence of 0.02 μg/ml of a CEFT peptide mixture for 24 hours. Different groups of treatment antibodies were added directly to the wells, mixed and further incubated for an additional 4 days. Cell culture supernatants were collected for multiplex cytokine measurement by MSD analysis.

### MSD Cytokine Analysis

IFN-γ, IL-10, IL-2 and TNF-α cytokine levels in the tissue culture supernatant were determined using MSD human V-Plex customized kits. Samples were first diluted 1:200 in Diluent 2. Calibrators were also prepared in Diluent 2 following the manufacturer's recommendations. Diluted samples and calibrators were added to black MSD plates which were subsequently sealed with an adhesive plate seal and incubated at room temperature with shaking for 2 hours. After adding 25 μL of the detection antibody solution, which was freshly prepared in Diluent 2 to each well, the plate was re-sealed and incubated at room temperature with shaking for another 2 hours. The plates were washed 3 times with 150 μL/well of PBS plus 0.05% Tween-20 before adding 150 μl/well of freshly diluted 2×read buffer and immediately read on a MESO QuickPlex reader. Data were analyzed using MSD Workbench software.

### Data Analysis

### MSD Data Analysis

MSD data was analyzed with Discovery Workbench software (MSD, version 4.0.9). Calibrators in the manufacturer's kit were included on each MSD plate to generate plate specific standard curves with R² value over 0.99 in all cases. The amounts of cytokine detected were back calculated based on the standard curve and the mean and standard deviation from three biological replicates were used to generate the graphs.

### Statistical Analysis

One-way ANOVA was performed on log-transformed, fold-change data over each treatment antibody's own isotype control. Dunnett's Multiple Comparison Test was performed to compare both mono-therapies vs. combination across different donors. P<0.05 was considered as statistical significant.

### Results

PBMC pre-stimulation assay development and test for combinatorial activity of H2L5 hIgG4PE with ipilimumab and pembrolizumab.

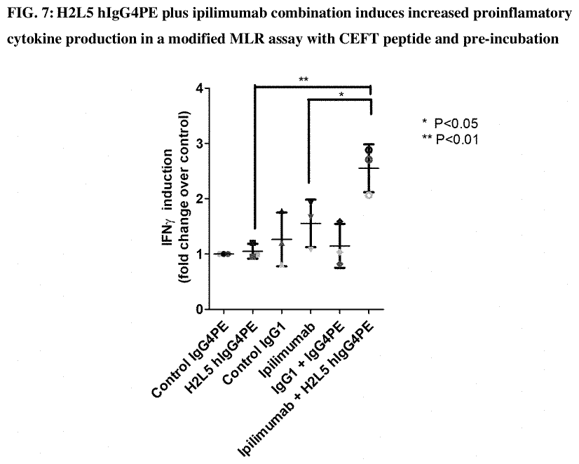

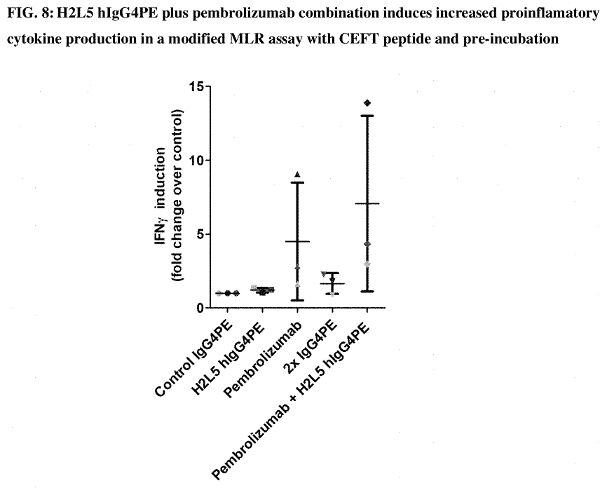

In order to determine the optimal conditions for pre-stimulation, human anti-CD3 Dynabeads and anti-CD3/CD28 Dynabeads (Thermo Fisher) were tested at different bead to cell ratios. After 48 hour pre-stimulation, cells were harvested and beads were magnetically removed prior to stimulation with anti-CD3 Dynabeads (bead to cell ratio=1:1) together with anti-ICOS antibody alone or in combination with anti-CTLA-4 or anti-PD1. H2L5 hIgG4PE single agent treatment resulted in induction of IFN-γ as compared to isotype control in all pre-stimulation conditions tested. The magnitude of IFN-γ induced by H2L5 hIgG4PE was inversely correlated with the strength of the pre-stimulation. The combination of H2L5 hIgG4PE together with ipilimumab demonstrated enhanced cytokine production as compared to either H2L5 HIGG4PE or ipilimumab alone in PBMCs that were weakly pre-stimulated. The combination effect was lost under plate-bound anti-CD3/anti-CD28 pre-stimulation conditions, which is considered a stronger pre-stimulation condition. Based upon these results, the pre-stimulation condition using anti-CD3/anti-CD28 beads at a bead to cell ratio of 1:20 was chosen for all future PBMC assays. Results from four individual donors are summarized for anti-CTLA-4 combination in FIG. 5 and combination with anti-PD-1 in FIG. 6.

H2L5 hIgG4PE Results in Dose-Dependent Cytokine Induction in a PBMC Pre-Stimulation Assay

The dose-dependent activity of H2L5 hIgG4PE was evaluated in human PBMCs pre-stimulated with anti-CD3/anti-CD28 beads at a pre-determined bead to cell ratio of 1:20. The anti-RSV IgG4PE and anti-ICOS 422.2 IgG1 Fc Disabled were included as controls. Eight concentrations of H2L5 HIGG4PE were tested (100, 30, 10, 3, 1, 0.3, 0.1, and 0.03 μg/ml). IFN-γ, IL-10 and TNF-α were evaluated by MSD in the tissue culture supernatants of PBMC samples. H2L5 hIgG4PE, but not isotype control IgG4 or Fc-Disabled 422.2, induced IFN-γ, IL-10 and TNF-α production in a dose-dependent manner. These results were used to determine the concentration of H2L5 hIgG4PE to be used in combination studies.

### Human MLR Assay Development

In an effort to optimize a human MLR assay, in addition to co-culture of human T cells and monocyte-derived immature DCs from a different donor, anti-CD3 beads were also added into the wells to provide a basal TCR stimuli to help prime the cells. Results demonstrated that anti-CD3 beads greatly increased the range of IFN-γ induction. Although ipilimumab alone can induce IFN-γ production in the absence of anti-CD3 beads, H2L5 hIgG4PE alone or the H2L5 HIGG4PE/ipilimumab combination only showed enhanced IFN-γ production over corresponding controls in the presence of anti-CD3 beads.

### Combinatorial Activity of H2L5 HIGG4PE and Ipilimumab in a Human MLR Assay

The immunostimulatory activity of H2L5 hIgG4PE alone or in combination with ipilimumab was tested in an allogeneic human MLR assay in which T cells that were pre-incubated with monocyte-derived immature DCs from an unmatched donor in the presence of 0.02 μg/ml CEFT peptides for 1 day. The H2L5 hIgG4PE/ipilimumab combination resulted in a significant enhancement in IFN-γ production as compared to either agent alone. Results were consistent across three donor pairs tested; however, modest variability was observed between donors (FIG. 7).

Combinatorial Activity of H2L5 hIgG4PE and Pembrolizumab in a Human MLR Assay

The combination of H2L5 hIgG4PE and pembrolizumab was also tested in the human allogeneic MLR assay described above. H2L5 hIg G4PE was tested alone and in combination with pembrolizumab at 10 μg/ml. The combination of H2L5 hIg G4PE and pembrolizumab resulted in increased IFN-γ as compared to either agent alone. However, statistical significance was not reached due to high donor variability and significant activity of single agent anti-PD-1 treatment in some donors (FIG. 8).

### Discussion

ICOS is a costimulatory receptor that is weakly expressed on naïve T cells and quickly upregulated in activated CD4⁺ and CD8⁺ T cells. The ligand for ICOS is ICOS-L (B7h, B7RP-1, CD275), which is expressed by professional APCs and by peripheral epithelial and endothelial cells following TNF-α stimulation. The ICOS:ICOS-L pathway provides a key costimulatory signal for T-cell proliferation and function. Due to its role in sustaining T-cell activation and effector functions, targeting ICOS by agonist antibodies could be a plausible approach to enhance anti-tumor immunity

Studies have shown an increase the frequency of ICOS^{hi} CD4⁺ effector T cells after CTLA-4 blockade by ipilimumab in several cancer models. In addition, upon CTLA-4 blockade, this cell population produced greater levels of INF-γ than ICOS^{lo} CD4⁺ T cells. In fact, the increase in the frequency of ICOS+ CD4 T cells has been identified as a pharmacodynamic biomarker of ipilimumab treatment in cancer patients. Studies, in wild-type C57BL/6 mice, demonstrated 80 to 90% tumor rejection follow CTLA-4 blockade therapy; however, in ICOS or ICOSL knockout mice the efficacy was decreased to less than 50%. The important role played by ICOS in the effectiveness of CLTA-4 blockade suggests that stimulating the ICOS pathway during anti-CTLA-4 therapy might increase therapeutic efficacy. Therefore, we set out to evaluate the combination activity of H2L5 hIgG4PE and ipilimumab.

Programmed cell death-1 (PD-1) was reported in 2000 to be another immune checkpoint molecule. The expression of PD-L1 (B7-H1), which is one of the ligands of PD-1, can be found on many cell types including T cells, epithelial cells, endothelial cells, and tumor cells. Antibodies targeting the PD-1/PD-L1 axis have also shown clinical responses in multiple tumor types. The FDA recently approved pembrolizumab and nivolumab as second generation of the immune checkpoint blockers for the treatment of cancer. Merck's pembrolizumab was shown to lead to response rates of ˜37 to 38% in patients with advanced melanoma, with a subsequent study reporting an overall response rate of 26% in patients who had progressive disease after prior ipilimumab treatment. Nivolumab, the anti-PD-1 antibody from BMS, also showed clinical benefit in patients with metastatic melanoma with a response rate of 40% and an overall survival rate of 72.9% at 1 year. In addition, nivolumab was also FDA-approved for advanced or metastatic non-small cell lung cancer. As the PD-1 checkpoint blockade antibodies become the dominant cancer immune therapy in the clinic, it will be important to evaluate H2L5 hIgG4PE in combination with an anti-PD-1 antibody for their combined anti-tumor activity.

Previously, a PBMC activation assay was developed and used to evaluate the T cell stimulation activity of a panel of anti-ICOS agonist antibodies. The data generated from those studies supported the candidate selection of clone 422.2 with an IgG4PE isotype as H2L5 hIgG4PE. In the previous assay, PBMC cells were pre-stimulated with plate bound anti-CD3 antibody at 1 μg/ml and anti-CD28 antibody at 3 μg/ml for 48 hours before they were harvested and re-stimulated with anti-CD3 and soluble ICOS antibodies that were being investigated. H2L5 hIgG4PE was shown to induce IFN-γ production in a dose-dependent manner. In order to determine the optimal conditions for pre-stimulation, human anti-CD3 Dynabeads and anti-CD3/CD28 Dynabeads (Thermo Fisher) were tested at different bead to cell ratios. Stimulation by beads is considered to be more physiological and the strength of the stimulation can be controlled more easily by constructing different bead to cell ratios. After 48 hours of pre-stimulation, cells were harvested and beads were magnetically removed prior to stimulation with anti-CD3 Dynabeads (bead to cell ratio=1:1) together with anti-ICOS antibody alone or in combination with anti-CTLA-4. The results showed that H2L5 hIgG4PE single agent treatment resulted in IFN-γ induction relative to isotype control in all pre-stimulation conditions tested. The magnitude of IFN-γ induced by H2L5 hIgG4PE was inversely correlated with the strength of the pre-stimulation. The combination of H2L5 hIgG4PE together with ipilimumab demonstrated enhanced cytokine production as compared to either H2L5 hIgG4PE or ipilimumab alone in PBMCs that were weakly pre-stimulated. The combination effect was lost under plate-bound anti-CD3/anti-CD28 pre-stimulation conditions, which is considered a stronger pre-stimulation condition. Based upon these results, the pre-stimulation condition using anti-CD3/anti-CD28 at a bead to cell ratio of 1:20 was chosen for all the future PBMC assays. H2L5 hIgG4PE and ipilimumab combination demonstrated a statistically significant increase in IFN-γ production as compared to either antibody treatment alone.

In the assay optimization effort, with an anti-CD3/anti-CD28 stimulation bead to cell ratio fixed at 1:20, the anti-CD3 beads used during the re-stimulation step were titrated down from bead to cell ratios of 1:1 to 1:3 and 1:10. The results showed that the lower the re-stimulation strength yielded lower the IFN-γ induction by H2L5 hIgG4PE. The combination effect by H2L5 hIgG4PE and ipilimumab was totally lost under re-stimulation at a bead to cell ratio of 1:3 and 1:10. Therefore, the re-stimulation anti-CD3 bead to cell ratio of 1:1 was kept for all future experiments.

With the pre-stimulation and re-stimulation conditions optimized, this assay was used to evaluate the dose response of H2L5 hIgG4PE. A total of 8 antibody concentrations were tested, which were 100, 30, 10, 3, 1, 0.3, 0.1 and 0.03 μg/ml. The anti-RSV IgG4PE and anti-ICOS 422.2 IgG1 Fc Disabled, the Fc Disabled version of H2L5 hIgG4PE, were used as controls. Results showed that H2L5 hIgG4PE, but not isotype control IgG4 or Fc-Disabled 422.2, induced IFN-γ, IL-10 and TNF-α production in a dose-dependent manner. It is interesting that the Fc Disabled version of H2L5 hIg G4PE exhibited a limited cytokine induction response, indicting the Fc receptor engagement is crucial for the T cell agonizing function of H2L5 hIg G4PE. These results were also used to determine the dose of H2L5 hIg G4PE for combination studies.

A mixed lymphocytes reaction (MLR) assay was also developed to evaluate the combination effect of H2L5 hIg G4PE and checkpoint blocking antibodies. MLR assay is an ex vivo cellular immune assay in which primary monocyte-derived immature dendritic cells (iDCs) were mixed with T cells isolated from a different donor. The mismatch of major histocompatibility complex (MHC) molecules on the surface of iDC cells can initiate T cell stimulation in an allogeneic setting. In the clinic, the MLR assay is well-known for identifying the compatibility of tissue transplants between donors and recipients.

In order to develop the MLR assay, fresh human monocytes were cultured in medium supplemented with human recombinant GM-CSF and IL-4 for a week to induce an immature DC phenotype. Then fresh human T cells from a different donor were isolated and mixed with the iDC cells at a 10:1 ratio (T:iDC). H2L5 hIg G4PE and ipilimumab mono-therapy or combinational treatments were added to the T cell/iDC co-culture in the presence or absence of anti-CD3 beads. The purpose of the anti-CD3 beads was to provide a basal TCR stimulus to help prime the T cells. Results showed anti-CD3 beads greatly increased the range of IFN-γ induction in the assay. Although ipilimumab alone can induce IFN-γ production in the absence of anti-CD3 beads, H2L5 hIgG4PE alone or the H2L5 hIgG4PE/ipilimumab combination showed enhanced IFN-γ production over corresponding controls in the presence of anti-CD3 beads. This result suggests that, in this assay, the TCR stimulus by DC cells alone may not be sufficient to induce ICOS expression on the surface of resting T cells that were freshly isolated from PBMCs. In order to improve the situation, a 24 hour iDC and T cells pre-incubation step was added before the addition of therapeutic antibodies. The CEFT peptide mix was also added into the assay procedure to better prime the T cells and to elicit an antigen-specific response. The CEFT peptide pool consists of 27 peptides selected from defined HLA class I and II-restricted T-cell epitopes from human Cytomegalovirus (HHV-5; CMV), Epstein-Barr virus (HHV-4; EBV), Influenza A and Clostridium tetani. Considering the high vaccination frequency against Influenza and Clostridium tetani and the high prevalence of CMV and EBV in the general population, recall antigen responses were expected for a majority of the human samples. The results showed that increased IFN-γ production was observed when T cells were pre-incubated with iDC cells for 24 hours, and the IFN-γ production further increased when CEFT peptides were added to the co-culture system. The immunostimulatory activity of H2L5 hIgG4PE alone or in combination with ipilimumab was tested in the allogeneic human MLR assay in which T cells that were pre-incubated with monocyte-derived immature DCs from an unmatched donor in the presence of 0.02 μg/ml CEFT peptides for 1 day. The H2L5 hIgG4PE/ipilimumab combination resulted in a significant enhancement in IFN-γ production as compared to either agent alone. The results were consistent across three donor pairs tested; however, modest variability was observed between donors.

Similarly, the combination of H2L5 hIgG4PE and pembrolizumab was also tested in the human allogeneic MLR assay described above. H2L5 hIgG4PE was tested alone and in combination with pembrolizumab at 10 μg/ml. The combination of H2L5 hIgG4PE and pembrolizumab resulted in increased IFN-γ as compared to either agent alone. However, statistical significance was not reached due to high donor variability and significant activity of single agent anti-PD-1 treatment in some donors.

In summary, these studies demonstrated the superior combination activity of H2L5 hIgG4PE with two FDA-approved check point inhibitors, ipilimumab and pembrolizumab, when compared to mono-therapies in two human immune cell based assays. In the studies reported here, H2L5 hIgG4PE was shown to promote T cell activation and T_{H}1 skewing (e.g. IFN-γ production) that is characteristic of productive anti-tumor immune responses.

### Example 3: Functional Activity of H2L5 hIgG4PE Alone and in Combination with Anti-PD1 and Anti-CTLA-4 Antibodies In Vivo

### Human PBMC Mouse Tumor Model

### Methods

### Experimental Preparations

All procedures on animals were reviewed and approved by the GSK Institutional Animal Care and Use Committee prior to initiation of the studies protocol.

### Preparation of Cell Lines:

A2058 were propagated according to ATCC protocol.

### Materials:

A2058 human melanoma cell line: ATCC, Cat #CRL-11147, lot #59349362
DPBS: ATCC, Cat #30-2200, Lot #63357436
Dulbecco's Modified Eagle's Medium: ATCC, Cat #30-2002, Lot #62596471 Expiration: October 2015
Fetal Bovine Serum: Sigma-Aldrich, Cat #12176c-1000 ml, lot #13G180R0H1, Expiration: July 2018
0.25% (w/v) Trypsin-0.53 mM EDTA: ATCC, Cat #30-2102, Lot #62420300
Antibiotic-Antimycotic (100×): Life Technologies, Cat #15240-062
T175 cell culture flask: Greiner bio-one, Cat #661175
T75 cell culture flask: Greiner bio-one, Cat #658175

### Medium:

A2058 complete growth medium: Dulbecco's Modified Eagle's Medium+10% FBS. Culture conditions: Atmosphere: Air, 95%; 5% carbon dioxide (CO2); Temperature: 37° C.
Upon receipt of the cells:
Pre-warm complete medium at 37° C.
Thaw the cells quickly in 37° C. water bath. Wipe the tube with 70% ethanol and transfer cells to 15 ml tube filled with prewarmed complete medium.
Centrifuge at 1200 rpm for 5 minutes to collect the cell pellet.
Add the cells back to T75 flask filled with prewarmed complete medium and incubate at 37° C.

### Subculture of the Cells:

Volumes are given for a 75 cm² flask (For T175 cm² flask, adjust the amount of dissociation and culture medium needed proportionally).
Remove and discard culture medium.
Briefly rinse the cell layer with DPBS to remove all traces of serum that contains trypsin inhibitor.
Add 2.0 to 3.0 mL of Trypsin-EDTA solution to flask and observe cells under an inverted microscope until cell layer is dispersed (2-3 minutes).
Note: To avoid clumping do not agitate the cells by hitting or shaking the flask while waiting for the cells to detach. Cells that are difficult to detach may be placed at 37° C. to facilitate dispersal.
Add 10 mL of complete growth medium and aspirate cells by gently pipetting.
Centrifuge at 1200 rpm for 5 minutes to collect the cell pellet, add 10 ml of complete growth medium
Add appropriate aliquots of the cell suspension to new culture vessels. Incubate cultures at 37° C.
Medium Renewal: Every 2 to 3 days

### Preparation of Tumor Cells for Mice Inoculation:

Wash cells with 1×DPBS, add 3 ml 1×Trypsin for 2-3 minutes.
Add complete growth media and collect the cell suspension in sterile conical centrifuge tube in the tissue culture hood.
Centrifuge the cells at 1200 rpm for 5 minutes to obtain cell pellet.
Wash cells with 1×DPBS solution, Centrifuge at 1200 rpm for 5 minutes to obtain cell pellet. Repeat the washing 2 times.
Count the cells by hemocytometer for cell number and viability.
Resuspend the cells in ice-cold PBS at concentrations for In Vivo inoculation (A2058, 2.5e7/ml, 2.5e6/100 μl/mouse).

### Tumor Cell Line Inoculation to NSG Mice

### Materials:

Mouse: NOD.Cg-Prkdcscid Il2rgtm1Wjl/SzJ. The Jackson Laboratory Stock: 005557 Female Age: 6 weeks
1 mL Tuberculin Syringes with Attached Needle 25 G 5/8: Becton Dickinson, Cat #305554
PDI™ Alcohol Prep Pads: Professional Disposables, Cat #B339
PDI™ Povidone-Iodine Prep Pad: Professional Disposables,
Cat #B40600
Preparation of mice
Mice should be 6 weeks old.
Allow 3-5 days acclimatization period after mice have arrived.
Shave the mice on right hind flank

### Preparation of the Injection

Clean and sterilize the inoculation area of the mice with iodine followed by ethanol pad
Use 1-cc syringe and a 25-gauge needle
Pull out the plunger, mix cells and add 100 ul of cells to the back of syringe, carefully insert the plunger.
Inject cells subcutaneously (s.c.) into the right hind flank of the mouse.
Tumor Growth Assessment
To measure a tumor, wet fur with 70% ethanol to make it easier to find tumor margins. Measure tumor size and body weight every 2-3 days.
Tumor size is measured with a digital caliper, and the volume is determined as follow: Tumor volume (mm3)=(length)×(width)/2
Human PBMC Intravenous Administration
Human PBMC administration can start 1 week after when the tumors have reached an average volume of approximately 100 mm3

### Materials:

Fresh Human PBMC: Alicells, cat #C-PB102-3B2
1 mL Tuberculin Syringes with Attached Needle 25 G 5/8: Becton Dickinson, Cat #305554
PDI™ Alcohol Prep Pads: Professional Disposables, Cat #B339
PDP Povidone-Iodine Prep Pad: Professional Disposables, Cat #B40600
Gauze sponges: Covidien, cat #441211
Mouse Tail Illuminator Restrainer: Braintree scientific, cat #MTI STD
PBMC preparation
Fresh human PBMC are purchased from Allcells by overnight shipment.
Centrifuge the cells at 1400 rpm for 5 minutes to obtain cell pellet.
Wash cells with 1×DPBS solution, Centrifuge at 1400 rpm for 5 minutes to obtain cell pellet.
Resuspend the cells in ice-cold PBS at concentrations for In Vivo injection (20e7/ml).
Use 1-cc syringe and a 25-gauge needle.
Pull out the plunger, mix cells and add 100 ul of cells to the back of syringe, carefully insert the plunger.
Keep the cells on ice.
Tail Vein injection
Warm the mice with an incandescent lamp for 5 minutes
Restrain the mice with tail illuminator restrainer.
Rotate the tail slightly to visualize vein.
Clean and sterilize injection site with iodine followed by ethanol pad
Insert needle into the vein at a slight angle and inject the cells.
Remove the needle and apply gentle compression with Gauze sponges until bleeding has stopped.
Return animals to their cage and observe for 5-10 minutes to make sure that bleeding has not resumed.

### Therapeutic Antibody Administration

1-3 days post human PBMC injection, mice are administrated with antibodies by Intraperitoneal injection.

### Materials:

Fully human IgG1 isotype control: Eureka therapeutics, cat #ET-901 (preclinical grade)
Lot #15-726 Expiration: February 2017
Ipilimumab (Yervoy): Bristol-Myers Squibb NDC 0003-2327-11, lot #921873 Expiration: April 2015; lot #4H69490, Expiration: May 2016
Fully human IgG4 isotype control: Eureka therapeutics, cat #ET-904 (preclinical grade) Lot #15-726 Expiration: February 2017
Anti human ICOS H2L5 hIgG4PE
Pembrolizumab (Keytruda): Merck, NDC 0006-3026-02, lot #L010592, Expiration: Apr. 26 2016
Intraperitoneal injection:
Draw up, into the syringe and needle, 100 μl of to be administered.
Line the bevel of the needle with the numbers on the syringe.
Sufficiently restrain the animal with your non-dominant hand.
Entry point for the needle: Draw an imaginary line across the abdomen just above the knees, the needle will be inserted along this line on the animal's right side and close to the midline. As this is a female, you can see that the point of entry is cranial to and slightly medial of the last nipple.
Tilt the mouse with its head slightly toward the ground so that its head is lower than its hind end.
Insert the needle into the abdomen at about a 30-degree angle.
The shaft of the needle should enter to a depth of about half a centimeter.
After injection, withdraw the needle and return the mouse to its cage.
Blood and tumor sampling
Materials:
Microvette CB300 (Serum): Braintree Scientific, Cat #MV-CB300 16440
Microvette CB300 (Hematology/Potassium EDTA): Braintree Scientific, Cat #MV-CB300 16444
Blood:
Mice were tail vein bled once a week.
30 μl of blood was collected in Microvette CB300 (Hematology/Potassium EDTA) for flow cytometry analysis.
Another 30 μl of blood was collected in serum Microvette CB300 and incubated for 2 hours at room temperature to allow clotting, followed by centrifugation at 2000×g in order to collect serum. Serum was stored at −20 until further analysis.

### Tumor:

Mice were euthanized when tumor size reached 2000 mm³. Tumors were collected and processed in the following procedure.

### Experimental Design

All studies were prepared according to procedures listed above.

H2L5 hI2G4PE Dose Response

This study was designed to determine dose-dependent activity of H2L5 hIgG4PE in human PBMC engrafted NSG mice implanted with A2058 melanoma tumors. Nine groups with 10 mice per group and 1 control group (Tumor only no PBMC) with 7 mice were assigned into each study. A summary of the treatment regimen for dose response using human PBMC from donor #7129 is present in Table. H2L5 hIgG4PE was dosed at 0.04, 0.4, 1.2 and 4 mg/kg. Ipilimumab was dosed at 3 mg/kg and an Fc-Disabled variant of the anti-ICOS agonist was tested at 1 mg/kg. Test groups were evaluated relative to the vehicle and matched isotype control groups. Survivability analysis concluded on day 49 at termination of the study.

TABLE 2
Summary of Treatment Regimen for H2L5 hIgG4PE
Dose Response in Mice

| | | | # mice/ | |
|---|---|---|---|---|
| Groups | Treatment 1 | Treatment 2 | group | Dosing |
| 1 | Tumor + | Vehicle | 10 | Twice |
| | huPBMC | | | weekly |
| | (donor #7129) | | | for 3 |
| | | | | weeks |
| 2 | Tumor + | human IgG1 | 10 | Twice |
| | huPBMC | Isotype | | weekly |
| | (donor #7129) | (3 mg/kg) | | for 3 |
| | | | | weeks |
| 3 | Tumor + | Ipilimumab | 10 | Twice |
| | huPBMC | (3 mg/kg) | | weekly |
| | (donor #7129) | | | for 3 |
| | | | | weeks |
| 4 | Tumor + | human IgG4 | 10 | Twice |
| | huPBMC | (4 mg/kg) | | weekly |
| | (donor #7129) | | | for 3 |
| | | | | weeks |
| 5 | Tumor + | H2L5 hIgG4PE | 10 | Twice |
| | huPBMC | (0.04 mg/kg) | | weekly |
| | (donor #7129) | | | for 3 |
| | | | | weeks |
| 6 | Tumor + | H2L5 hIgG4PE | 10 | Twice |
| | huPBMC | (0.4 mg/kg) | | weekly |
| | (donor #7129) | | | for 3 |
| | | | | weeks |
| 7 | Tumor + | H2L5 hIgG4PE | 10 | Twice |
| | huPBMC | (1.2 mg/kg) | | weekly |
| | (donor #7129) | | | for 3 |
| | | | | weeks |
| 8 | Tumor + | H2L5 hIgG4PE | 10 | Twice |
| | huPBMC | (4 mg/kg) | | weekly |
| | (donor #7129) | | | for 3 |
| | | | | weeks |
| 9 | Tumor + | ICOS-Fc- | 10 | Twice |
| | huPBMC | disabled | | weekly |
| | (donor #7129) | (1 mg/kg) | | for 3 |
| | | | | weeks |
| 10 | Tumor | Untreated | 7 | Twice |
| | (no PBMC) | | | weekly |
| | (donor #7129) | | | for 3 |
| | | | | weeks |

Efficacy and Pharmacodynamic (PD) Activity Study with H2L5 hIgG4PE in Combination with Ipilimumab and Pembrolizumab

### Study Objectives:

To evaluate the anti-tumor activity of H2L5 hIgG4PE monotherapy dosed at 0.04 mg/kg and 0.4 mg/kg.

To evaluate the anti-tumor activity of H2L5 hIgG4PE dosed in combination with ipilimumab or pembrolizumab with matched isotype controls.

Collection of tissue for future pharmacodynamic activity study of H2L5 hIgG4PE. A total of 22 treatment groups with 10 mice per group were assigned to this study. Groups 1-16 were the efficacy cohorts and 17-22 were pharmacodynamic activity cohorts.

For combination treatments, H2L5 hIgG4PE (0.04 or 0.4 mg/kg) and ipilimumab or IgG1 (3 mg/kg) or H2L5 hIgG4PE (0.04 or 0.4 mg/kg) and pembrolizumab or IgG4 (5 mg/kg) were dosed. H2L5 hIgG4PE and ipilimumab as well as the matched isotype controls were dosed twice weekly for 6 doses, pembrolizumab and isotype control were dosed every 5 days until end of the H2L5 hIgG4PE dose. For the pharmacodynamic tissue collection cohorts, H2L5 hIgG4PE was dosed at 0.004, 0.04, 0.4 and 1.2 mg/kg. Treatment groups were evaluated relative to the vehicle and isotype control groups. Treatment groups for vehicle, isotypes and H2L5 hIgG4PE alone and in combination with ipilimumab and pembrolizumab using human PBMC from donor number #6711 are shown in Table. Analysis concluded on day 59 at termination of the study.

TABLE 3
Treatment groups of mice in A2058 melanoma tumor model

| | | | # | |
|---|---|---|---|---|
| | | | mice/ | |
| Group | Treatment 1 | Treatment 2 | group | Dosing |
| 1 | Tumor + | Vehicle | 10 | Twice a week for 6 doses |
| | huPBMC | | | |
| | (donor | | | |
| | #6711) | | | |
| 2 | Tumor + | Isotype control (IgG1 | 10 | IgG1 Twice a week for 6 doses |
| | huPBMC | 3 mg/kg + IgG4 5 mg/kg) | | IgG4 every 5 days until the end of |
| | (donor | | | ICOS dose |
| | #6711) | | | |
| 3 | Tumor + | Ipilimumab 3 mg/kg + | 10 | Twice a week for 6 doses |
| | huPBMC | IgG4 5 mg/kg | | IgG4 every 5 days until the end of |
| | (donor | | | ICOS dose |
| | #6711) | | | |
| 4 | Tumor + | Pembrolizumab 5 mg/kg + | 10 | IgG1 Twice a week for 6 doses |
| | huPBMC | IgG1 3 mg/kg | | Pembrolizumab every 5 days until |
| | (donor | | | the end of ICOS dose |
| | #6711) | | | |
| 5 | Tumor + | H2L5 hIgG4PE | 10 | IgG1 and ICOS Twice a week for |
| | huPBMC | 0.04 mg/kg + IgG1 | | 6 doses |
| | (donor | 3 mg/kg | | |
| | #6711) | | | |
| 6 | Tumor + | H2L5 hIgG4PE 0.4 | 10 | IgG1 and ICOS Twice a week for |
| | huPBMC | mg/kg + IgG1 3 mg/kg | | 6 doses |
| | (donor | | | |
| | #6711) | | | |
| 7 | Tumor + | Ipilimumab 3 mg/kg + | 10 | Ipilimumab Twice a week for 6 |
| | huPBMC | Pembrolizumab | | doses Pembrolizumab every 5 days |
| | (donor | 5 mg/kg | | until the end of ICOS dose |
| | #6711) | | | |
| 8 | Tumor + | H2L5 hIgG4PE 0.04 | 10 | Ipilimumab and ICOS Twice a |
| | huPBMC | mg/kg + Ipilimumab | | week for 6 doses |
| | (donor | 3 mg/kg | | |
| | #6711) | | | |
| 9 | Tumor + | H2L5 hIgG4PE 0.4 | 10 | Ipilimumab and ICOS Twice a |
| | huPBMC | mg/kg + Ipilimumab | | week for 6 doses |
| | (donor | 3 mg/kg | | |
| | #6711) | | | |
| 10 | Tumor + | H2L5 hIgG4PE 0.04 | 10 | ICOS Twice a week for 6 doses |
| | huPBMC | mg/kg + Pembrolizumab | | Pembrolizumab every 5 days until |
| | (donor | 5 mg/kg | | the end of ICOS dose |
| | #6711) | | | |
| 11 | Tumor + | H2L5 hIgG4PE 0.4 | 10 | ICOS Twice a week for 6 doses |
| | huPBMC | mg/kg + Pembrolizumab | | Pembrolizumab every 5 days until |
| | (donor | 5 mg/kg | | the end of ICOS dose |
| | #6711) | | | |
| 12 | Tumor + | IgG4 5 mg/kg | 10 | Twice a week for 6 doses |
| | huPBMC | | | |
| | (donor | | | |
| | #6711) | | | |
| 13 | Tumor + | Pembrolizumab | 10 | Pembrolizumab every 5 days until |
| | huPBMC | 2.5 mg/kg | | the end of ICOS dose |
| | (donor | | | |
| | #6711) | | | |
| 14 | Tumor + | Pembrolizumab 5 mg/kg | 10 | Pembrolizumab every 5 days until |
| | huPBMC | | | the end of ICOS dose |
| | (donor | | | |
| | #6711) | | | |
| 15 | Tumor + | H2L5 hIgG4PE 0.4 | 10 | ICOS Twice a week for 6 doses |
| | huPBMC | mg/kg | | |
| | (donor | | | |
| | #6711) | | | |
| 16 | Tumor + | H2L5 hIgG4PE 0.4 | 10 | ICOS Twice a week for 6 doses |
| | huPBMC | mg/kg + Pembrolizumab | | Pembrolizumab every 5 days until |
| | (donor | 5 mg/kg + Ipi | | the end of ICOS dose |
| | #6711) | | | |
| 17 | Tumor + | Vehicle | 10 | Twice a week for pharmacodynamic |
| | huPBMC | | | activity, 5 mice harvested 24 hr post |
| | (donor | | | 2nd dose and 5 mice harvested |
| | #6711) | | | 24 hr post 4nd dose |
| 18 | Tumor + | Isotype Control (IgG4) | 10 | Twice a week for pharmacodynamic |
| | huPBMC | 1.2 mg/kg | | activity, 5 mice harvested 24 hr post |
| | (donor | | | 2nd dose and 5 mice harvested |
| | #6711) | | | 24 hr post 4nd dose |
| 19 | Tumor + | H2L5 hIgG4PE 0.004 | 10 | Twice a week for pharmacodynamic |
| | huPBMC | mg/kg | | activity, 5 mice harvested 24 hr post |
| | (donor | | | 2nd dose and 5 mice harvested |
| | #6711) | | | 24 hr post 4nd dose |
| 20 | Tumor + | H2L5 hIgG4PE 0.04 | 10 | Twice a week for pharmacodynamic |
| | huPBMC | mg/kg | | activity, 5 mice harvested 24 hr post |
| | (donor | | | 2nd dose and 5 mice harvested |
| | #6711) | | | 24 hr post 4nd dose |
| 21 | Tumor + | H2L5 hIgG4PE 0.4 | 10 | Twice a week for pharmacodynamic |
| | huPBMC | mg/kg | | activity, 5 mice harvested 24 hr post |
| | (donor | | | 2nd dose and 5 mice harvested |
| | #6711) | | | 24 hr post 4nd dose |
| 22 | Tumor + | H2L5 hIgG4PE 1.2 | 10 | Twice a week for pharmacodynamic |
| | huPBMC | mg/kg | | activity, 5 mice harvested 24 hr post |
| | (donor | | | 2nd dose and 5 mice harvested |
| | #6711) | | | 24 hr post 4nd dose |

Efficacy Study Evaluating H2L5 hIgG4PE Dosed in Combination with Ipilimumab or Pembrolizumab

This study was designed to evaluate the anti-tumor efficacy of H2L5 hIgG4PE (dosed at 0.01 and 0.04 mg/kg) in combination with ipilimumab or pembrolizumab with matched isotype controls in the human PBMC engrafted NSG mouse using A2058 melanoma tumor model. A total of 13 groups with 10 mice per group were assigned into the study. Group 2 was the combined isotype control of humanized IgG1 and IgG4. H2L5 hIgG4PE was dosed at 0.01 mg/kg (Group 12) and 0.04 mg/kg (Group 13) as single agent. For combination treatments, H2L5 hIgG4PE (0.01 and 0.04 mg/kg) and ipilimumab or IgG1 (3 mg/kg) or H2L5 hIgG4PE (0.01 and 0.04 mg/kg) and pembrolizumab or IgG4 (5 mg/kg) was dosed. H2L5 hIgG4PE and ipilimumab as well as the matched isotype controls were dosed twice weekly for 6 doses, pembrolizumab and isotype control was dosed every 5 days until end of the H2L5 hIgG4PE dose. A summary of treatment groups, using human PBMC from donor #4568, is presented in Table. Treatment groups were evaluated relative to the vehicle and isotype control groups. Survivability analysis was concluded on day 33 at termination of the study.

TABLE 4
Treatment groups of mice in A2058 melanoma tumor model

| | | | # | |
|---|---|---|---|---|
| | | | mice/ | |
| Group | Treatment1 | Treatment2 | group | Dosing |
| 1 | Tumor + | Vehicle | 10 | Twice a week for 6 doses |
| | huPBMC | | | |
| | (donor | | | |
| | #4568) | | | |
| 2 | Tumor + | Isotype control (IgG1 | 10 | IgG1 Twice a week for 6 doses |
| | huPBMC | 3 mg/kg + IgG4 5 mg/kg) | | IgG4 every 5 days until the end of |
| | (donor | | | ICOS dose |
| | #4568) | | | |
| 3 | Tumor + | Ipilimumab 3 mg/kg + | 10 | Twice a week for 6 doses IgG4 |
| | huPBMC | IgG4 5 mg/kg | | every 5 days until the end of ICOS |
| | (donor | | | dose |
| | #4568) | | | |
| 4 | Tumor + | Pembrolizumab 5 mg/kg + | 10 | IgG1 Twice a week for 6 doses |
| | huPBMC | IgG1 3 mg/kg | | Pembrolizumab every 5 days until |
| | (donor | | | the end of ICOS dose |
| | #4568) | | | |
| 5 | Tumor + | H2L5 hIgG4PE 0.01 | 10 | IgG1 and ICOS Twice a week for |
| | huPBMC | mg/kg + IgG1 | | 6 doses |
| | (donor | 3 mg/kg | | |
| | #4568) | | | |
| 6 | Tumor + | H2L5 hIgG4PE 0.04 | 10 | IgG1 and ICOS Twice a week for |
| | huPBMC | mg/kg + IgG1 | | 6 doses |
| | (donor | 3 mg/kg | | |
| | #4568) | | | |
| 7 | Tumor + | Ipilimumab 3 mg/kg + | 10 | Ipilimumab Twice a week for 6 |
| | huPBMC | Pembrolizumab | | doses Pembrolizumab every 5 days |
| | (donor | 5 mg/kg | | until the end of ICOS dose |
| | #4568) | | | |
| 8 | Tumor + | H2L5 hIgG4PE 0.01 | 10 | Ipilimumab and ICOS Twice a |
| | huPBMC | mg/kg + Ipilimumab | | week for 6 doses |
| | (donor | 3 mg/kg | | |
| | #4568) | | | |
| 9 | Tumor + | H2L5 hIgG4PE 0.04 | 10 | Ipilimumab and ICOS Twice a |
| | huPBMC | mg/kg + Ipilimumab | | week for 6 doses |
| | (donor | 3 mg/kg | | |
| | #4568) | | | |
| 10 | Tumor + | H2L5 hIgG4PE 0.01 | 10 | ICOS Twice a week for 6 doses |
| | huPBMC | mg/kg + Pembrolizumab | | Pembrolizumab every 5 days until |
| | (donor | 5 mg/kg | | the end of ICOS dose |
| | #4568) | | | |
| 11 | Tumor + | H2L5 hIgG4PE 0.04 | 10 | ICOS Twice a week for 6 doses |
| | huPBMC | mg/kg + Pembrolizumab | | Pembrolizumab every 5 days until |
| | (donor | 5 mg/kg | | the end of ICOS dose |
| | #4568) | | | |
| 12 | Tumor + | H2L5 hIgG4PE | 10 | Twice a week for 6 doses |
| | huPBMC | 0.01 mg/kg | | |
| | (donor | | | |
| | #4568) | | | |
| 13 | Tumor + | H2L5 hIgG4PE 0.04 | 10 | Twice a week for 6 doses |
| | huPBMC | mg/kg | | |
| | (donor | | | |
| | #4568) | | | |

### Statistical Analysis

The event for survival analysis was tumor volume >2000 mm³, tumor ulceration, mouse body weight loss >20%, moribund or found dead, whichever came first. The exact time to cut-off volume was estimated by fitting a linear line between log tumor volume and day of two observations, the first observation that exceed the cut-off volume and the one observation that immediately preceded the cut-off volume. Kaplan-Meier (KM) method was carried out to estimate the survival probability of different treatment groups at a given time. The median time to endpoint and its corresponding 95% confidence interval was reported. Whether or not KM survival curves are statistically different between any two groups was then tested by log-rank test.

Tumor volume data from the last day in which there were 10 animals per group (i.e. before any animals were euthanized) was utilized to make tumor volume comparisons between the different treatment groups. Prior to the analysis, the tumor volume was natural log transformed due to the inequality of variance in the different treatment groups. ANOVA followed by pair-wise comparison were then carried out on the log transformed data.

Graphpad Prism software was used to plot the tumor growth and body weight data.

### Results

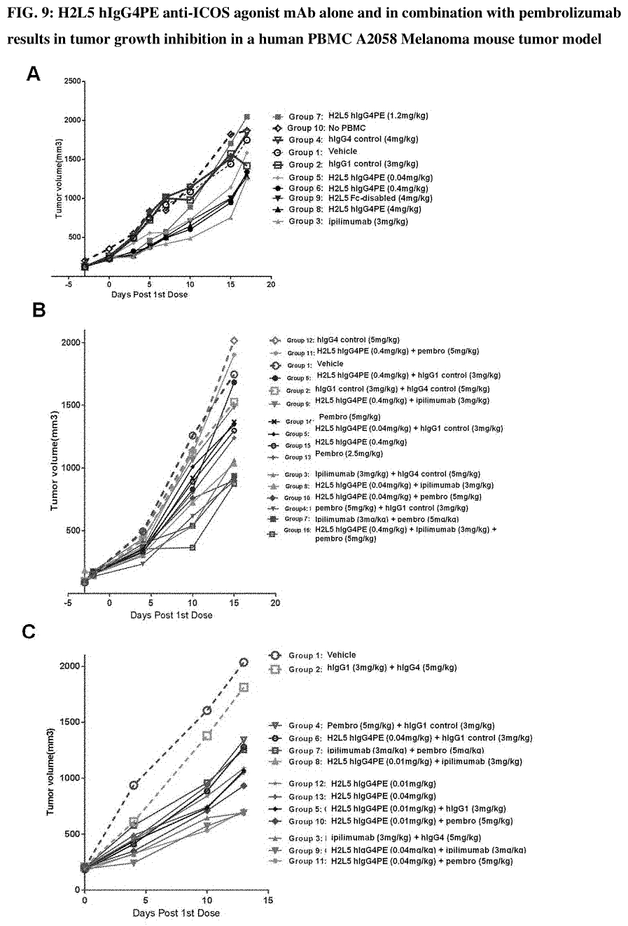

H2L5 hIgG4PE Dose Response (FIG. 9A)

### Tumor Growth Inhibition:

Control group: Human PBMC (donor 7129) showed no effect on A2058 tumor growth in NSG mice. A2058 tumor bearing mice with or without human PBMC, A2058 tumor bearing mice with human PBMC treated with vehicle and isotype control antibodies developed tumors that progressed as expected (Group #1 vs. Group #10, Group #1 vs. Group #2, Group #1 vs. Group #4, p=1).

Ipilimumab treatment at 3 mg/kg (Group #3) demonstrated significant tumor growth inhibition (p<0.03) as compared to vehicle control Group #1, however the statistical significance was lost (p<0.22) when compared to the isotype control Group #2. This indicated the isotype antibody may affect tumor growth.

H2L5 hIgG4PE treatment at 0.4 mg/kg demonstrated a trend of tumor growth inhibition and increased survivability of mice compared to other doses, although the affects were not statistically significant when compared to either vehicle or isotype control.

### Clinical Observations:

Loss of body weight in mice was observed during the study which was approximately 20% at the end of study. It has been reported that both GvHD and tumor burden can result in a drop in mice body weight, though in this study the body weight loss seemed to be more related to A2058 tumor since tumor bearing mice without PBMC engraftment (group #10) showed the same trend. Tumor ulceration was observed in multiple tumors during the study, including the isotype control group.

### Mouse Fates:

Most mice were removed upon tumors reaching volumes >2000 mm³. Three mice were euthanized due to tumor ulceration, and three mice were euthanized due to body weight loss of >20%. Nine mice were found dead randomly across the groups, including two in the vehicle, and three total in the isotype control groups. These deaths were attributed to the susceptibility of the model for a Graft-versus-Host Disease state, and not treatment related since no pattern was observed with treatment groups compared to vehicle or isotype control groups.

Efficacy study with H2L5 hIgG4PE in combination with ipilimumab and Pembrolizumab (FIG. 9B)

### Tumor Growth Inhibition:

Control group: A2058 tumor bearing mice with human PBMC treated with vehicle or isotype control antibodies developed tumors which grew as expected.

### Monotherapy:

Ipilimumab treatment at 3 mg/kg combined with IgG4 (Group #3) resulted in significant tumor growth inhibition (p<0.04) as compared to vehicle control Group #1. However, when compared to isotype control Group #2, the statistical significance was lost (p<0.23).

Pembrolizumab treatment alone at 2.5 or 5 mg/kg (Group #13, 14) showed observable tumor growth inhibition without statistical significance when compared to vehicle or isotype control group #12. Pembrolizumab combined with IgG1 (Group #4), showed observable tumor growth inhibition without statistical significance, however a significant increase in survival was observed (p<0.04) as compared to vehicle control Group #1. Statistical significance was lost (p<0.4) when compared with isotype control Group #2.

H2L5 hIgG4PE treatment alone at 0.4 mg/kg (Group #15) showed observable tumor growth inhibition without statistical significance as compared to vehicle or isotype control group #12. H2L5 hIgG4PE at 0.04 or 0.4 mg/kg combined with IgG1 (Group #5 and 6) showed observable delay in tumor progression and mice survival but didn't reach statistical significance.

### Combination Treatment:

Combination of H2L5 hIgG4PE (0.04 or 0.4 mg/kg) with ipilimumab (3 mg/kg). Groups #8 and #9 showed no additional tumor growth inhibition as compared to Ipilimumab alone (Group #3). Combination of H2L5 hIgG4PE (0.04 or 0.4 mg/kg) with pembrolizumab (5 mg/kg) Groups #10 and #11 demonstrated modest but insignificant tumor growth inhibition and mice survival compared to pembrolizumab monotherapy, Group #4, or H2L5 hIgG4PE monotherapy Groups #5 and #6.

### Clinical Observations:

Mice body weight loss observed during the study was approximately 20%. Tumor ulceration was apparent in multiple tumors during the study across the majority of group.

### Mouse Fates:

A total of 100 out of 160 mice were euthanized when tumor volumes reached >2000 mm³. 29 mice were euthanized due to tumor ulceration, 18 mice were found dead, 12 mice were euthanized due to body weight loss >20%, and one mouse was euthanized as moribund. Mice were found dead across the groups including the isotype control group #2. These deaths were attributed to the susceptibility of the model for a Graft-versus-Host Disease state, and not treatment related since no pattern was observed with treatment groups compared to the isotype control group.

Efficacy study evaluating H2L5 hIgG4PE dosed in combination with ipilimumab or pembrolizumab (FIG. 9C)

### Tumor Growth Delay:

Control group: A2058 tumor bearing mice with human PBMC treated with vehicle or isotype control antibodies developed tumors which grew as expected.

### Monotherapy:

Ipilimumab treatment at 3 mg/kg combined with IgG4 (Group #3) demonstrated significant tumor growth inhibition (p<0.02) and significant increase in survival (p<0.01) as compared to vehicle control Group #1. Compared to isotype control Group #2 however, the tumor growth inhibition did not reach significance (p<0.13) while significant increase in mice survival remained (p<0.04).

Pembrolizumab treatment at 5 mg/kg combined with IgG1 (Group #4) showed tumor growth inhibition without statistical significance as compared to vehicle or isotype control Group #2.

H2L5 hIgG4PE treatment alone at 0.01 mg/kg or 0.04 mg/kg (Group #12 and #13) demonstrated significant tumor growth inhibition (p<0.03) compared to vehicle control group #1 H2L5 hIgG4PE dosed at 0.04 mg/kg also showed a significant increase in mice survival (p<0.048) as compared to vehicle control group #1. However, as compared to isotype control group #2, tumor growth inhibition and survival did not reach statistical significance for groups #12 and #13. H2L5 hIgG4PE at 0.01 mg/kg combined with IgG1 (Group #5) showed significant tumor growth inhibition (p<0.03) and mice survival (p<0.03) as compared to vehicle control group #1. However, as compared to isotype control group #2, tumor growth delay and survival did not reach statistical significance. H2L5 hIgG4PE at 0.04 mg/kg combined with IgG1 (Group #6) showed observable tumor growth inhibition and mice survival, but did not reach statistical significance.

### Combination Treatment:

The combination of H2L5 hIgG4PE with ipilimumab (0.01 mg/kg plus ipilimumab 3 mg/kg; Group #8) showed observable tumor growth inhibition and mice survival but failed to reach statistical significance. H2L5 hIgG4PE combination with ipilimumab (0.04 mg/kg plus ipilimumab 3 mg/kg; Group #9) demonstrated significant tumor growth inhibition (p<0.00) and a significant increase in mice survival (p<0.04) as compared to vehicle control group #1 or isotype control group #2 (p<0.02). However, as compared to isotype control survival failed to reach statistical significance. Combination activity did not reach significance as compared to monotherapy ipilimumab group #3 or H2L5 hIgG4PE monotherapy groups.

H2L5 hIgG4PE (0.01 mg/kg or 0.04 mg/kg) combination with pembrolizumab (5 mg/kg), Groups #10 and #11, showed significant tumor growth inhibition. (p≤0.03) and significant increase of mice survival observed (p<0.03) when comparing to vehicle control group #1. When comparing to isotype control group #2, the tumor growth inhibition significance remained in the 0.04 mg/kg H2L5 hIgG4PE combination with pembrolizumab (p<0.03). The survival benefit failed to reach statistical significance however. The combination failed to reach significance as compared to either monotherapy treatment group pembrolizumab group #3 or H2L5 hIgG4PE group #5 or #6. Thus, H2L5 hIgG4PE combined with pembrolizumab (0.01 or 0.04 mg/kg plus pembrolizumab 5 mg/kg) demonstrated an increase in tumor growth inhibition and mice survival but failed to reach statistical significance versus isotype control or monotherapies.

### Clinical Observations:

Mice body weight loss observed during the study was approximately 20%. Tumor ulceration was observed across the majority of groups during the study.

### Mouse Fates:

A total of 91 mice were euthanized due to tumor size >2000 mm³, 34 mice were euthanized due to tumor ulcerations, and 5 mice were found dead. These deaths were attributed to the susceptibility of the model for a Graft-versus-Host Disease state.

### Discussion

Efficacy of H2L5 hIgG4PE as a monotherapy and in combination with pembrolizumab as well as ipilimumab was evaluated in the human PBMC engrafted NSG mouse model with A2058 melanoma tumors. This model where human PBMC are intravenously injected into adult immunodeficient NSG (NOD/SCID/IL-2Rγnull) mice is known as the Hu-PBMC NSG model. It induces a Graft-versus-Host Disease (GvHD) and has been used to study effector and memory T cell activity. The Hu-PBMC NSG model was implanted with human cancer cell line A2058 subcutaneously to investigate the effect of human immunotherapeutic antibodies on tumor growth. The limitations of this model include onset of GvHD symptoms, loss of body weight, and frequent tumor ulcerations which prevent survival monitoring for longer period of time as is possible with syngeneic mouse tumor models.

Initial studies evaluating H2L5 hIgG4PE at doses ranging from 0.04 mg/kg to 4 mg/kg showed that doses in the lower range demonstrated modest tumor growth inhibition. Delay in tumor progression and increased survival of mice was observed in dose groups ranging from 0.04 to 0.4 mg/kg though not statistically significant when compared to the isotype control groups. Based on these studies, H2L5 hIgG4PE doses of 0.04 to 0.4 mg/kg were selected for further evaluation alone and in combination with pembrolizumab and ipilimumab in two studies with PBMC grafts from two different donors (donor numbers 4568 and 6711). Modest responses for H2L5 hIgG4PE monotherapy and combination with pembrolizumab were observed in one of the two combination studies performed. The combination study using PBMC donor 4568 (Table 4, FIG. 9C) demonstrated anti-tumor activity of the monotherapy and combination while the study using PBMC donor 6711 (Table 3, FIG. 9B) did not show significant anti-tumor effect, which likely was a result of donor PBMC differences between studies, which reflect the patient response variability that may be observed in the clinic. In this second combination study with PBMC donor 4568, enhanced tumor growth inhibition and increased survivability of mice was observed in the combination group when compared to either agent alone, although this difference was not statistically significant. Combination synergy was observed however, since the H2L5 hIgG4PE 0.04 mg/kg dose in combination with pembrolizumab 5 mg/kg resulted in a statistically significant decrease in tumor volume ten days post first dose and increased survivability versus the isotype control group (p<0.05), while the monotherapies did not. In fact, 50% of the mice in the H2L5 hIgG4PE and pembrolizumab combination group remained on study by day 33, but were removed due to tumor ulcerations. Only four mice were removed from study due to tumor volume from this combination group, while 8 to 9 mice were removed from study in the pembrolizumab and isotype groups.

Anti-PD1 therapy did not demonstrate statistically significant activity in this model as seen with the limited change in tumor growth and survival seen with pembrolizumab treated cohort compared to isotope treated cohort. Ipilimumab monotherapy showed a trend of tumor growth inhibition modestly better than pembrolizumab in both studies, and it showed statistically significant increase in survival versus isotype in the second combination study with the responsive PBMC donor 4568 (p≤0.04). The H2L5 hIgG4PE 0.01 mg/kg dose in combination with ipilimumab 3 mg/kg showed a significant increase in survival versus ipilimumab (p≤0.02), but not versus H2L5 hIgG4PE monotherapy. There were no additional significant effects on tumor volume observed with the combination of H2L5 hIgG4PE and ipilimumab in this model compared to either agent alone. Mice from across all treatment groups including vehicle and isotype control groups were found dead as reported in the Fate Tables. These deaths were attributed to the susceptibility of the model for a Graft-versus-Host Disease state, and not treatment related.

### Example 4: Functional Activity of Anti-Murine ICOS Agonist Antibody Alone and in Combination with Anti-PD1 and Anti-CTLA-4 Antibodies In Vivo

### CT26 and EMT6 Syngeneic Mouse Tumor Models

### CT26 Murine Colon Carcinoma Mouse Tumor Model

### Methods

This study was conducted under a protocol which was approved by the GSK Institutional Animal Care and Use Committee prior to commencement of the study.

### Animals

In this study 164 female BALB/c mice from Harlan Sprague Dawley. Mice were 6-8 weeks old at the beginning of the study when they were inoculated.

### Cell Culture and Inoculation

One vial of CT-26 cells (ATCC: CRL-2638) (3×10⁶ cells; P-11) was thawed from −140° C. and plated in RPMI with 10% FBS. Cells were subcultured 3 times over 10 days. Trypsin/EDTA was used to facilitate cell detachment from culture flask during subculturing. Cells were collected, washed twice, and re-suspended in RPMI without FBS at 5×10⁵ cells/ml. Mice were inoculated subcutaneously with 0.1 ml cells (5×10⁴ cells/mouse) on the right hind flank.

On the day of cell collection and inoculation, cell counts were done on Beckman Coulter Vi-cell XR and checked by hemacytometer. Cells were detached from flask with trypsin/EDTA and washed twice, first with RPMI+10% FBS and second with RPMI only and resuspended in 10 ml RPMI. 178×10⁶ cells were collected in 20 ml RPMI with 98.8% viability. 1.685 ml cell suspension (15×10⁶ cells total) was added to 28.315 ml RPMI.

15×10⁶ cells/30 ml media=5×10⁵ cells/ml. This equates to 5×10⁴ cells/100 ul.

### Antibody Formulation and Preparation

Antibodies were diluted from stock source vials to desired concentrations in sterile 0.9% saline on the day of dosing. Anti-ICOS agonist clone C398.4 was tested at 0.05 mg/kg and 0.5 mg/kg. Each dose was also tested with both anti-PD1 10 mg/kg and anti-CTLA-4 1 mg/kg.

### Experimental Protocol(s)

### Tumor Monitoring and Dosing

Mice were inoculated on day 0. On day 11 body weight and tumor volume were measured. Mice were randomized into the 12 study groups shown in

Table with 10 mice/group based on tumor size. Randomization was done using Studylog Study Director software. Mice were dosed based on the study design chart twice weekly starting on randomization day and continuing for 6 total doses. Dosing was interperitoneal (IP) in 100 ul volume of 0.9% saline vehicle. Tumor volume and body weight were measured 3 times per week throughout the study.

### Endpoints

Mice were removed from the study for tumor burden when tumor volume was greater than 2000 mm³. Tumor volume was calculated by applying length and width caliper measurements to the following formula: TV=0.52*L*W².

Additionally mice were removed from study when tumors developed open ulcerations. Ulcerations were observed throughout the experiment, however scabbed over ulcerations alone were not an endpoint unless they formed open holes.

Although it did not apply to any mice in this study a third endpoint established at the beginning of the study was a decrease of 20% body weight.

### Drugs and Materials

| Antibody | Vendor | Catalog # | Lot | Clone |
|---|---|---|---|---|
| ICOS | Biolegend | 93108 | B205973 | C398.4 |
| PD1 | BioXcell | BE0146 | 5792-10/0815B | RMP1-14 |
| CTLA-4 | BioXcell | BE0164 | 5632-4/0715 | 9D9 |
| Mouse IgG2b | BioXcell | BE0086 | 4700/1014 | MCP-11 |
| Rat IgG2a | BioXcell | BE0089 | 5679-6/0815 | 2A3 |
| Hamster IgG | Biolegend | 92257 | B205974 | HTK888 |

All antibodies were diluted to desired concentrations in 0.9% saline and saline was used as a vehicle control.

Data Analysis

The event for survival analysis is tumor volume of 2000 mm³ or tumor ulceration, whichever came first. The exact time to cut-off volume was estimated by fitting a linear line between log tumor volume and day of two observations, the first observation that exceed the cut-off volume and the one observation that immediately preceded the cut-off volume. The Kaplan-Meier (KM) method was carried out to estimate the survival probability of different treatment groups at a given time. The median time to endpoint and its corresponding 95% confidence interval was reported. Whether or not KM survival curves are statistically different between any two groups was then tested by the log-rank test.

Tumor volumes at 17 days after initial dosing between the different treatment groups were compared. Prior to the analysis, the tumor volume was natural log transformed due to the inequality of variance in the different treatment groups. ANOVA followed by pair-wise comparison were then carried out on the log transformed data.

TABLE 5
Study Groups

| Group | |
|---|---|
| No. | Treatment |
| 1 | Saline |
| 2 | Mouse IgG2b 20 ug + |
| | Hamster IgG 10 ug |
| 3 | Rat IgG2a 200 ug + |
| | Hamster IgG 10 ug |
| 4 | Hamster IgG 10 ug |
| 5 | ICOS 1 ug |
| 6 | ICOS 10 ug |
| 7 | CTLA-4 20 ug |
| 8 | PD1 200 ug |
| 9 | ICOS 1 ug + CTLA-4 20 ug |
| 10 | ICOS 10 ug + CTLA-4 20 ug |
| 11 | ICOS 1 ug + PD1 200 ug |
| 12 | ICOS 10 ug + PD1 200 ug |

The raw p-value, as well as the false discovery rate (FDR) adjusted p-values, from the comparisons of days to events by survival analysis and the comparisons of log transformed tumor volume at day 10 between treatment groups are shown in the above table. Comparisons, using FDR adjusted p-values ≤0.05, are declared to be statistically significant.

### Results

Mouse fate tracking showed that the number of mice removed from study for tumor burden and tumor ulceration. All remaining mice are tumor free at study day 61 except 1 mouse in G7 which has a tumor volume of 579.16 mm³.

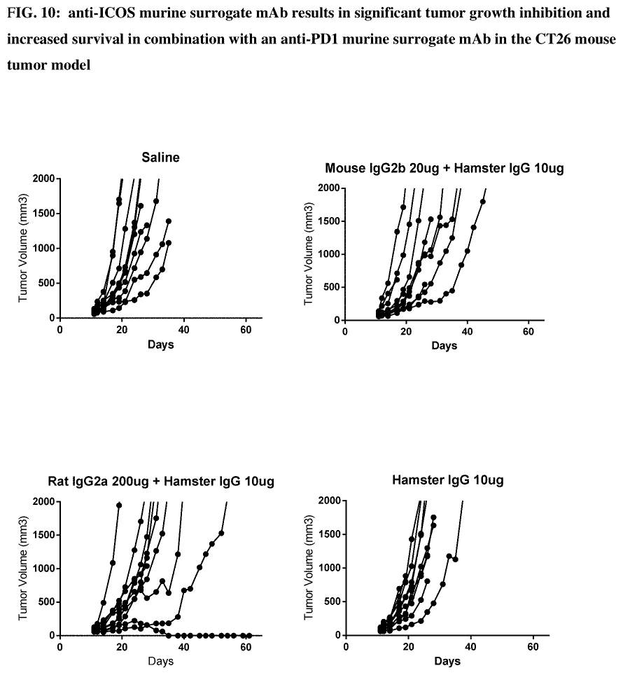

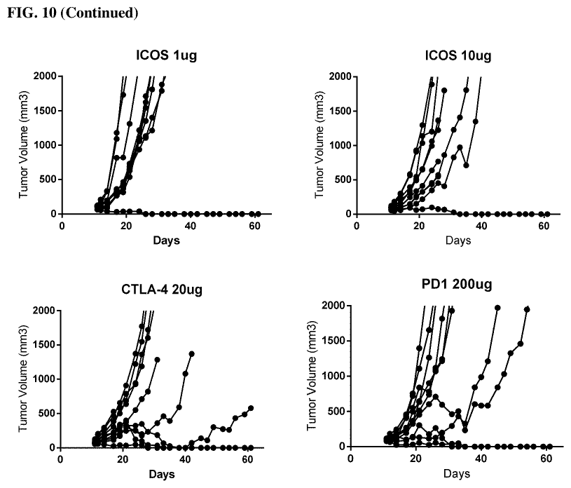

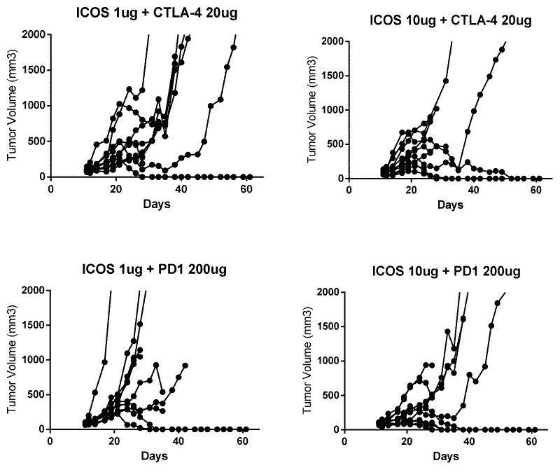

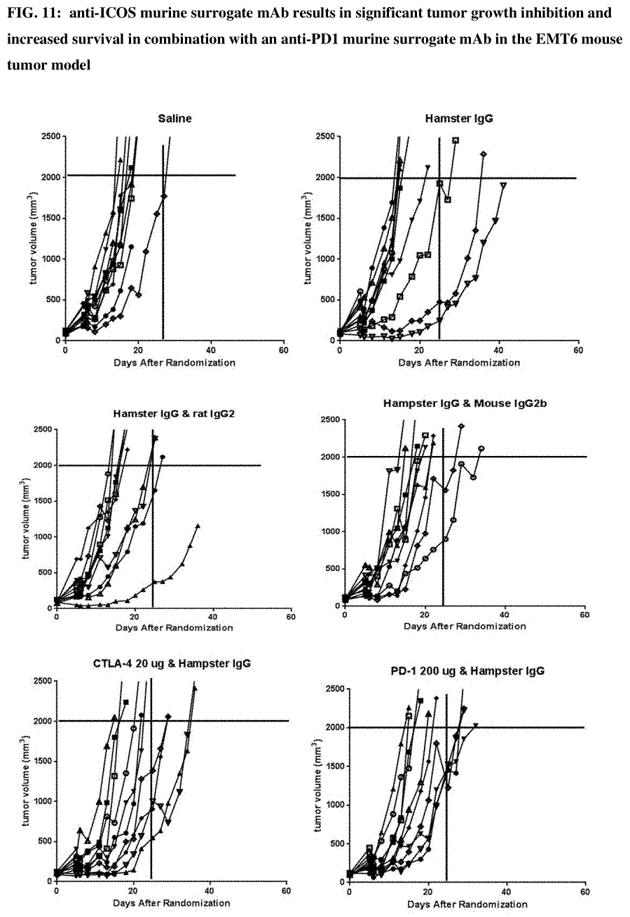

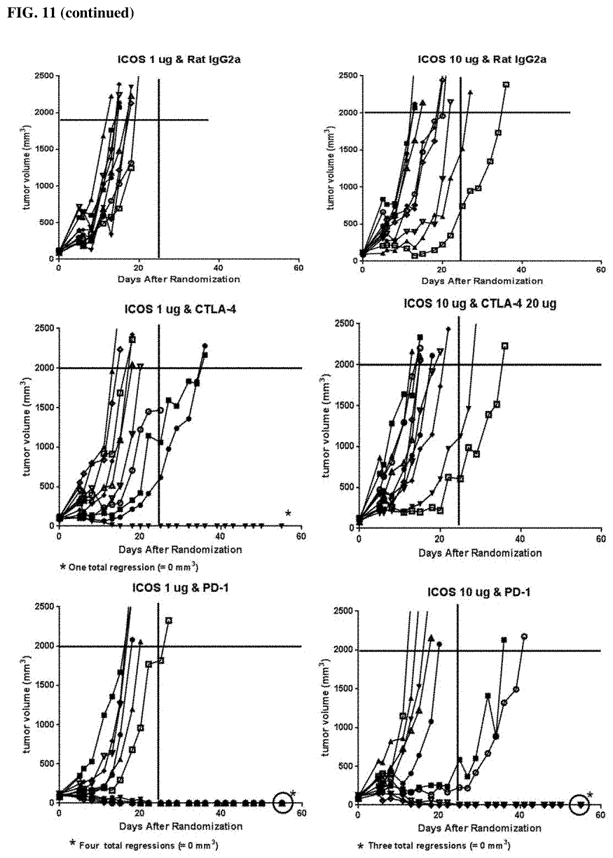

For survival (time to endpoints) groups 9 and 12 showed significant increase in survival compared to the vehicle control group (p=0.008 and p=0.001 respectively). Group 12 showed statistically significant extended survival compared to groups 2, 4, and 5 (p=0.006, 0.001, 0.02). However, no combination group showed statistically significant (p<0.05) increased survival over either monotherapy. (FIG. 10)

### Discussion

The combination therapy groups, particularly the high dose anti-ICOS and anti-PD1 combination (Group 12), demonstrated tumor growth inhibition and increased survival over monotherapy and isotype control groups, although statistical significance was not reached at Day 61. The isotype control for group 12 was the Rat IgG2a+Hamster IgG group 3. The monotherapy groups for comparison are; ICOS 10 ug (group 6) and PD1 200 ug (group 8). A total of 5 mice remained as tumor free in group 12 compared to 1 in group 3, 1 in group 6 and 1 in group 8. The survival benefit was quantified by taking the day each mouse reached any of the pre-determined study endpoints. A number of mice were removed from study for open tumor ulcerations and not due to tumor burden.

In the high dose ICOS+ CTLA-4 combination group (group 10) an increased number of mice were removed due to tumor ulceration by day 31 which likely masked the survival and anti-tumor benefit that this combination provided. In this group, 5 mice were removed for tumor ulcerations and only 2 for tumor burden reaching 2000 mm³. All tumors removed due to tumor ulceration where still at modest size when taken off study, and it is expected that tumor ulceration may have been the result of a therapy-induced anti-tumor immune response in these mice. Three mice remained tumor free in this group out to day 61. The 2 mice removed for tumor burden were the lowest number of mice removed for tumor burden of all groups.

### EMT6 Mammary Carcinoma Mouse Tumor Model

### Experimental Protocol(s)

All procedures and euthanization criteria described in this document are in accordance with IACUC protocol AUP0606. Animals are weighed and inoculated on the right hind quarter with 100 ul of 1×10⁵ EMT6 tumor cells per mouse. The number of mice inoculated is equal to at least 130% of what was needed for the study. Assuming 30% failure rate (either too big or too small at time of start of study), the goal was to have n=10 for each group. After tumor cell inoculation, tumor growth and total body weight were measured 3 times a week with a Fowler “ProMax” digital caliper for 4 weeks or longer. Antibodies were acquired from a commercial vendor and diluted to desired concentration in 0.9% saline. Dosing (i.p.) occurred biweekly, for a total of 6 doses and initiated on the day of randomization, designated as Day 0, when average tumor volume approximated 100 mm³, approximately 7 to 8 days after inoculation. Randomization was performed using the Studylog Study Director Suite software. Length and width of tumors was measured in order to determine tumor volume using the formula (tumor volume=L*W²*0.52). Tumor measurement of greater than 2,000 mm³ for an individual animal resulted in removal from study. Mice may also be removed from the study due to weight loss (>20%), tumor ulceration, or any other obvious inhibition of normal mouse activity due to morbidity.

In this study, a total of 191 animals were inoculated with EMT6 cells in order to generate enough mice with tumors in the desired size range for 13 groups of 10 mice each as shown in Table. Saline vehicle injected mice and isotype control groups served as controls for ICOS, PD1 and CTLA-4 mAb treated mice. The isotype control for ICOS (Hamster IgG) was dosed at 10 ug alone and in combination with the isotype for CTLA-4 (mouse IgG2b) or PD-1 (rat IgG2a). Monotherapy treatment groups for anti-CTLA-4 (9D9) and anti-PD-1 (RMP1-14) were dosed at 20 and 200 ug per mouse, respectively, and evaluated in combination with the ICOS isotype control. The C398.4 clone of ICOS agonist was dosed at 10 and 1 ug per mouse. Efficacy of the ICOS agonist was also evaluated at 10 and 1 ug per mouse dosed in combination with anti-CTLA-4 or anti-PD-1. An additional group of PD-1 and CTLA-4 at predescribed concentrations was included as a positive control comparator group. Statistical analysis of tumor volume was performed on day 13 post randomization. Survivability analysis included mice on study through day 60.

TABLE 6
Study Groups

| Dosing | treatment 1 | treatment 2 | n = |
|---|---|---|---|
| Group 1: | saline | | 10 |
| 1 × 10⁵ cells per | | | |
| Group 2: | Hamster IgG 10 μg | mIgG2b 20 μg | 10 |
| 1 × 10⁵ cells per | | | |
| Group 3: | Hamster IgG 10 μg | rIgG2a 200 μg | 10 |
| 1 × 10⁵ cells per | | | |
| Group 4: | Hamster IgG 10 μg | | 10 |
| 1 × 10⁵ cells per | | | |
| Group 5: | ICOS 10 μg | | 10 |
| 1 × 10⁵ cells per | | | |
| Group 6: | ICOS 1 μg | | 10 |
| 1 × 10⁵ cells per | | | |
| Group 7: | CTLA4 20 μg | Hamster IgG 10 μg | 10 |
| 1 × 10⁵ cells per | | | |
| Group 8: | PD-1 200 μg | Hamster IgG 10 μg | 10 |
| 1 × 10⁵ cells per | | | |
| Group 9: | ICOS 10 μg | CTLA4 20 μg | 10 |
| 1 × 10⁵ cells per | | | |
| Group 10: | ICOS 1 μg | CTLA4 20 μg | 10 |
| 1 × 10⁵ cells per | | | |
| Group 11: | ICOS 10 μg | PD-1 200 μg | 10 |
| 1 × 10⁵ cells per | | | |
| Group 12: | ICOS 1 μg | PD-1 200 μg | 10 |
| 1 × 10⁵ cells per | | | |
| Group 13: | CTLA4 20 μg | PD-1 200 μg | 10 |
| 1 × 10⁵ cells per | | | |

### Drugs and Materials

### Animals

Female Balb/c mice from 6 to 8 weeks of age were received from Harlan Sprague Dawley and housed in accordance with IACUC standards.

### EMT6 Cells

EMT6 cells were thawed and cultured in cell culture flasks for eight days prior to inoculation. Cells were passed 3 times in this time. On the day of inoculation, the cells are harvested from the flask in complete medium. Cells are centrifuged and resuspended in Weymouth's (with 15% FBS). This step is repeated 3 times in Weymouth's media without FBS. Cell density and viability are checked via trypan blue exclusion. Cells are then diluted to desired density (1×10⁶ cells per mL).

### Immunotherapeutics

All therapeutics were diluted to desired concentrations in 0.9% sodium chloride on the day of dosing and injected i.p. using a 30 G needle. Therapeutic and control dilutions are presented below in Table.

TABLE 7
Therapeutic dilutions

| | starting | desired | | dose/ | | Volume | add | Total | total |
|---|---|---|---|---|---|---|---|---|---|
| | conc. | conc. | dilution | mouse | number | needed | stock | diluent | volume |
| Rx | mg/mL | mg/mL | 1: | mg | of mice | mL | mL | mL | mL |
| mouse | 4.46 | 0.1 | 44.6 | 0.02 | 10 | 2 | 0.10 | 4.36 | 4.46 |
| IgG2b | | | | | | | | | |
| rat IgG2a | 6.92 | 1 | 6.92 | 0.2 | 10 | 2 | 0.40 | 2.37 | 2.77 |
| Hamster | 1.47 | 0.05 | 29.4 | 0.01 | 50 | 10 | 0.40 | 11.36 | 11.76 |
| IgG | | | | | | | | | |
| CTLA4 | 6.1 | 0.1 | 61 | 0.02 | 40 | 8 | 0.15 | 9 | 9.15 |
| PD-1 | 7.44 | 1 | 7.44 | 0.2 | 40 | 8 | 1.30 | 8.372 | 9.672 |
| ICOS | 5 | 0.05 | 100 | 0.01 | 30 | 6 | 0.10 | 9.9 | 10 |
| ICOS | 0.05 | 0.005 | 10 | 0.001 | 30 | 6 | 1.00 | 9 | 10 |

### Data Analysis

### Statistical Analysis

The event for survival analysis was tumor volume of 2000 mm³ or tumor ulceration, whichever came first. The exact time to cut-off volume was estimated by fitting a linear line between log tumor volume and day of two observations, the first observation that exceed the cut-off volume and the one observation that immediately preceded the cut-off volume. The Kaplan-Meier (KM) method was carried out to estimate the survival probability of different treatment groups at a given time. The median time to endpoint and its corresponding 95% confidence interval was reported. Whether or not KM survival curves were statistically different between any two groups was then tested by the log-rank test.

Tumor volumes at 13 days after initial dosing between the different treatment groups were compared. Prior to the analysis, the tumor volume was natural log transformed due to the inequality of variance in the different treatment groups. ANOVA followed by pair-wise comparison were then carried out on the log transformed data. SAS 9.3 and R 3.0.2 Analysis Software was utilized.

### Results

Balb/c mice were inoculated and randomized into groups of ten based on treatment regimen 8 days later. Administration of therapeutics or controls began on randomization day (Day 0) and continued twice a week for 3 weeks.

The saline treated group grew tumors at the expected rate relative to previous EMT-6 studies. All mice in the saline vehicle group were euthanized due to tumor size or ulceration by day 30. Treatment with hamster IgG alone or in combination with rat IgG2a or mouse IgG2b, resulted in no statistically significant change in average tumor growth or survival when compared to the saline vehicle group.

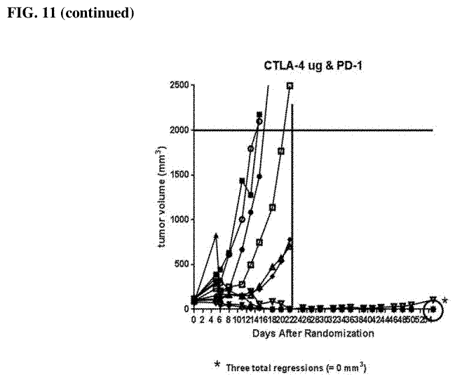

At 13 days post randomization, the ICOS monotherapy groups demonstrated little observable change in average tumor growth as compared to isotype controls. However, the high dose ICOS treatment group (10 g) demonstrated an apparent trend towards more tumor growth inhibition than the low dose group. An effect that was comparable to the CTLA-4 monotherapy activity was observed. Monotherapy treatment with PD-1 mAb also resulted in some observable, but statistically insignificant reduction in average tumor volume at day 13. However, as with ICOS and CTLA-4 monotherapy, this did not result in increased survival when compared to that of the appropriate isotype groups. Treatment with the combination of anti-PD-1 and anti-ICOS antibody clone C398.4 at the 10 ug dose resulted in considerable tumor growth inhibition as compared to control and monotherapy treatment groups (FIG. 11). Three mice in this combination group achieved complete tumor regression, a considerable improvement over control or monotherapy treatment groups. However, due to the statistical criteria used, statistically significant improvement in survival was not reached. The combination of anti-PD-1 with 1 ug of ICOS agonist antibody clone C398.4 did result in a statistically significant decrease in average tumor growth at day 13 as compared to saline vehicle control (p<0.05) and ICOS monotherapy (p<0.05) groups of 1 and 10 ug. Four mice from this treatment regimen achieved complete tumor regression resulting in significant trend towards increased survival that failed to reach statistical significance.

The ICOS antibody at both doses in combination with anti-CTLA-4 demonstrated little observable benefit in tumor growth inhibition or survival as compared to monotherapy treatment with either antibody.

### Discussion

While isotype controls resulted in no obvious change in average tumor volume or overall survival when compared to the saline vehicle group, there were individual animals in the hamster IgG group (group 4) and the hamster IgG and rat IgG2a (group 3) that demonstrated delayed tumor growth. In the hamster IgG & rat IgG2a isotype group, one mouse survived beyond the last saline vehicle mouse, being sacrificed on day 36 due to ulceration with a tumor that measured 1156.56 mm³ in volume. Two mice in the hamster IgG group survived longer than the saline group. One animal was euthanized due to tumor size on day 36, and the second one on day 41 due to ulceration with a measurement of 1899.28 mm³.

The dosing regimen of anti-PD-1 with 10 ug of anti-ICOS agonist led to an observable inhibition of tumor growth resulting in a decrease in tumor volume at day 13 when compared to isotype controls, although this difference was less obvious when compared to anti-PD-1 monotherapy. However, the combination did result in a total of five animals surviving beyond any in the anti-PD-1 monotherapy group, with three mice experiencing complete tumor regression as compared to none in the anti-PD-1 monotherapy group.

Pairing anti-PD-1 with a 1 ug dose of ICOS agonist antibody led to an observable decrease in average tumor size at day 13 when compared to isotype controls and respective monotherapy groups. This decrease was statistically significant when compared to saline vehicle control (p<0.05) and the 1 ug ICOS monotherapy group (p<0.05). Four mice experienced complete tumor regression and survived beyond any in the PD-1 monotherapy group

The survival benefit observed with the ICOS+PD1 combination group was not found to reach statistical significance relative to controls by day 60. However, the tumor growth inhibition and survival benefit of the ICOS+PD1 combination treatment groups was comparable to the activity observed with the PD1+CTLA-4 combination group, which was considered a positive control for anti-tumor activity in this study. This suggests that a combination of ICOS and PD1 antibodies may have benefit similar to CTLA-4 and PD1 combinations, which have demonstrated significant clinical activity in some tumor types.

Of the 130 mice enrolled in this study, 12 remained alive at day 60 with 11 having achieved complete tumor regression. Of the 118 mice that met endpoints for study removal, 111 were removed due to reaching a tumor size of 2000 mm³. The remaining seven mice were euthanized due to ulceration on the tumor. Occurrences of ulceration were spread out among the groups. Groups 1 (Saline), 3 (hamster IgG & rat IgG2a), 4 (hamster IgG), 6 (1 ug ICOS), and 10 (CTLA-4 with 1 ug ICOS) all had one animal removed due to ulceration. Group 13 (CTLA-4+PD-1) showed two animals sacrificed due to ulceration. The remaining groups had no animals removed due to ulceration.

1.-20. (canceled)

21. A method of treating cancer in a human in need thereof comprising administering ipilimumab and at least one additional agent directed to ICOS to the subject, wherein administration of ipilimumab increases ICOS expression on a T cell.

22. The method of claim 21, wherein the agent directed to ICOS is an agonist antibody directed to ICOS.

23. The method of claim 21, further comprising administering an anti-PD-1 antibody to said human.

24. The method of claim 23, wherein said anti-PD-1 antibody is selected from pembrolizumab and nivolumab.

25. The method of claim 24, wherein said anti-PD-1 antibody is pembrolizumab.

26. The method of claim 21, wherein ipilimumab and the agent directed to ICOS are administered to said human simultaneously or sequentially.

27. The method of claim 26, wherein ipilimumab and the agent directed to ICOS are administered to said human simultaneously.

28. The method of claim 26, wherein ipilimumab and the agent directed to ICOS are administered to said human sequentially.

29. The method of claim 21, wherein said cancer is selected from head and neck cancer, breast cancer, lung cancer, colon cancer, ovarian cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

30. The method of claim 21, further comprising administering at least one additional neo-plastic agent to said human.

31. The method of claim 30, wherein the at least one additional neo-plastic agent is a chemotherapeutic agent.

32. The method of claim 31, wherein the chemotherapeutic agent is docetaxel, methotrexate, paclitaxel, gemcitabine, fluorouracil, carboplatin, cisplatin or a combination thereof.

33. The method of claim 21, wherein administering ipilimumab increases ICOS expression on a T cell tumor infiltrating lymphocytes.

34. The method of claim 21, wherein administering ipilimumab increases ICOS expression on tumor infiltrating lymphocytes.

35. The method of claim 21, wherein administering ipilimumab increases expression of TNF-alpha, IL-12p70, IL-13, and IL-5 cytokines.

36. The method of claim 21, wherein the patient is in need of increased expression of ICOS.

37. The method of claim 21, wherein the cancer is head and neck cancer.

38. The method of claim 21, wherein the cancer is lung cancer.
